SEQUENCE LISTING (12 sequences)

SEQ ID NO: 1 - DNA, 15 bp, Artificial Sequence
  Description of Artificial Sequence Synthetic oligonucleotide
gaattcagagaataa

SEQ ID NO: 2 - protein, 4 aa, Artificial Sequence
  Description of Artificial Sequence Synthetic peptide
EFRE

SEQ ID NO: 3 - DNA, 27 bp, Artificial Sequence
  Description of Artificial Sequence Synthetic oligonucleotide
gaattccaccaccaccaccaccactaa

SEQ ID NO: 4 - protein, 8 aa, Artificial Sequence
  Description of Artificial Sequence Synthetic peptide
EFHHHHHH

SEQ ID NO: 5 - DNA, 63 bp, Artificial Sequence
  Description of Artificial Sequence Synthetic oligonucleotide
gaattccaccaccaccaccacgcgtctgcggccgcggctgcaggccaccaccaccaccactaa

SEQ ID NO: 6 - protein, 20 aa, Artificial Sequence
  Description of Artificial Sequence Synthetic peptide
EFHHHHHASAAAAAGHHHHH

SEQ ID NO: 7 - DNA, 48 bp, Artificial Sequence
  Description of Artificial Sequence Synthetic oligonucleotide
gaattcagagaaggcggcgctgcaggccaccaccaccaccaccactaa

SEQ ID NO: 8 - protein, 15 aa, Artificial Sequence
  Description of Artificial Sequence Synthetic peptide
EFREGGAAGHHHHHH

SEQ ID NO: 9 - protein, 14 aa, Artificial Sequence
  Description of Artificial Sequence Synthetic peptide
AEDQVDPRLIDGKS

SEQ ID NO: 10 - protein, 10 aa, Artificial Sequence
  Description of Artificial Sequence Synthetic peptide
HHHHHHHHHH

SEQ ID NO: 11 - protein, 6 aa, Artificial Sequence
  Description of Artificial Sequence Synthetic peptide
HHHHHH

SEQ ID NO: 12 - protein, 5 aa, Artificial Sequence
  Description of Artificial Sequence Synthetic peptide
HHHHH

(19) United States
(12) Patent Application Publication
(10) Pub. No.: US 2008/0260858 A1
(43) Pub. Date: Oct. 23, 2008

(54) Universal Procoagulant

(75) Inventors: James H. Morrissey, Champaign, IL (US); Emily Kerestes Waters, Jamaica Plain, MA (US)

Correspondence Address: EVAN LAW GROUP LLC, 600 WEST JACKSON BLVD., SUITE 625, CHICAGO, IL, 60661, US

(73) Assignee: The Board of Trustees of the University of Illnois, Urbana, IL (US)

(21) Appl. No.: 11/816,401

(22) PCT Filed: Feb. 10, 2006
(86) PCT No.: PCT/US2006/004789

§ 371 Date: Apr. 2, 2008

Related U.S. Application Data
(60) Provisional application No. 60/653,695, filed on Feb. 16, 2005.

(51) Int. Cl. A61K 33/34; C12Q 1/56; C07K 14/435; A61K 33/30; A61P 7/04

(52) U.S. Cl. 424/638; 435/13; 530/350; 424/646; 424/641

(57) ABSTRACT

A thromboplastin reagent comprises: (i) activated sTF, (ii) a metal-chelating lipid, (iii) a metal ion, and (iv) a phospholipid. Activated sTF preferably includes the extracellular domain of TF and an oligohistidine moiety having at least 2 histidine residues, more preferably 2-10 histidine residues. Preferably, the histidine residues are consecutive. Attaching a metal binding domain, such as an oligohistidine tag, to the C-terminus of sTF allows the protein to bind to phospholipid vesicles that contain metal-chelating lipid. Metal complexes of this activated sTF and metal-chelating lipids have all of the desirable expression, handling, and solubility characteristics of sTF, and exhibit procoagulant activities in plasma clotting tests that are comparable to relipidated rTF. In addition, it was discovered that, under some circumstances, Ni-lipids are themselves procoagulant, even in the absence of activated sTF. Further studies indicated that Ni-lipids are potent activators of the contact pathway of blood clotting.

### REFERENCE To RELATED APPLICATIONS

This application claims the benefit of U.S. Provisional Application No. 60/653,695 entitled “UNIVERSAL PROCOAGULANT” filed 16 Feb. 2005, the entire contents of which are hereby incorporated by reference, except where inconsistent with the present application.

### FEDERALLY SPONSORED RESEARCH OR DEVELOPMENT

This application was funded in part under the following research grants and contracts: National Institutes of Health (NHLBI) Grant No. R01 HL47104. The U.S. Government may have certain rights in this invention.

### BACKGROUND

A schematic of the clotting cascades, with only the clotting factors and Ca²⁺ listed, is shown in FIG. 15. In the figure the various clotting factors are indicated by their Roman numeral (i.e., factor VII is indicated by VII). The intrinsic pathway (also referred to as the contact pathway) of blood coagulation is initiated when contact is made between blood and certain artificial surfaces. The extrinsic pathway (also referred to as the tissue factor pathway) of blood coagulation is initiated upon vascular injury which leads to exposure of tissue factor (also identified as factor III). The dotted arrow represents a point of cross-over between the extrinsic and intrinsic pathways. The two pathways converge at the activation of factor X to Xa. Factor Xa has a role in the further activation of factor VII to VIIa. Active factor Xa hydrolyzes and activates prothrombin to thrombin. Thrombin can then activate factors XI, VIII and V, furthering the cascade. Ultimately, the role of thrombin is to convert fibrinogen to fibrin, which forms clots.

Tissue factor, a cell-surface protein, is responsible for triggering the blood clotting system in normal hemostasis and a variety of thrombotic diseases [1,2]. Tissue factor accomplishes this by tightly binding and allosterically activating coagulation factor VIIa (VIIa), a plasma serine protease. The 1:1 complex of tissue factor and VIIa (TF:VIIa) is the first enzyme in the tissue factor pathway of blood coagulation, with VIIa functioning as the catalytic subunit and tissue factor as the regulatory subunit. The clotting cascade is therefore triggered when TF:VIIa activates two plasma serine protease zymogens (coagulation factors IX and X) via limited proteolysis, ultimately leading to the formation of a hemostatic plug consisting of a fibrin clot and activated platelets.

Wild-type human tissue factor (TF) is a single polypeptide chain of 261 or 263 amino acids, containing four cysteine residues which form two disulfide bonds. It is a type I integral membrane protein, meaning that its N-terminus is located outside the cell, and its C-terminus is in the cytoplasm. TF has a single membrane-spanning domain that anchors the protein in the plasma membrane. The extracellular domain of TF is the portion that binds to, and allosterically activates, VIIa. The cytoplasmic domain of TF is dispensable for TF procoagulant activity, but membrane anchoring of TF is essential for full TF activity [3]. A truncated, soluble form of tissue factor that does not have the membrane-spanning domain nor the cytoplasmic domain (sTF) has been produced by recombinant means [3-7]. Unlike membrane-anchoring TF, sTF is highly water soluble [5,8]. While sTF retains the ability to bind to VIIa and allosterically activate it (as measured by hydrolysis of small, peptidyl-amide substrates), sTF has greatly reduced procoagulant activity [4, 5, 9, 10]. It has been shown that sTF is selectively deficient in supporting the conversion of zymogen factor VII to the active enzyme form, VIIa [9, 11]. (The ability to promote the conversion of factor VII to VIIa is one of the important functions of TF [12]. Loss of this function explains the low procoagulant activity of sTF with normal human plasma [9].) The unique deficiency of sTF has been exploited to create a clotting assay that quantifies plasma VIIa levels without interference from zymogen factor VII [13]. On the other hand, this deficiency in sTF procoagulant activity means that it cannot substitute for membrane-anchoring TF in standard clotting assays like the Prothrombin Time (PT) assay.

Because of its solubility properties, sTF is considerably easier to express, purify, and handle than is membrane-anchoring TF. In some expression systems used for making sTF, secretion of sTF is targeted to the periplasmic space of E. coli, an oxidizing environment that allows disulfide bonds to form [6]. sTF is easily released from the periplasmic space of E. coli by osmotic shock, and furthermore, sTF does not require any special conditions to maintain water solubility. sTF may be purified by immunoaffinity chromatography from E. coli releasates, taking advantage of a peptide epitope (HPC4 epitope) engineered onto the N-terminus of sTF [6]. In the presence of calcium ions, the sTF-HPC4 fusion protein binds with high affinity to immobilized HPC4 antibody. After washing the immunoaffinity beads, purified sTF is eluted using EDTA. Expression yields are approximately 20 mg sTF per liter of E. coli culture.

Recombinant, membrane-anchoring tissue factor (rTF) is expressed at much lower levels in E. coli cells than is sTF, and rTF is more difficult to handle at all stages of the purification process. The same targeting vector was used for E. coli expression of rTF that was used for sTF. (This targets the extracellular domain of rTF to the periplasmic space, while the membrane anchor remains embedded in the inner membrane of E coli.) Extraction of rTF from E. coli requires complete lysis of the bacteria. It also requires the use of detergents, both to extract rTF from the membrane and also to keep rTF solubilized. Purification of rTF is achieved using the same immunoaffinity chromatography method as sTF, except that a detergent (typically, 0.1% Triton X-100) is included in all of the solutions to which rTF is exposed. Expression yields of rTF in the E. coli expression system are approximately 1 mg per liter of E. coli culture, which is at least twenty-fold lower than the yield of sTF.

The Prothrombin Time (PT) test is widely used to monitor oral anticoagulation therapy by coumarins, as a general screening test for the blood clotting system, and as the basis for specific factor assays. Clotting times obtained with the PT test (PT time) are primarily dependent on the plasma levels of the vitamin K-dependent coagulation factors II (prothrombin), VII, and X, and on the levels of two non-vitamin K-dependent proteins, factor V and fibrinogen. Coumarin treatment antagonizes the vitamin K carboxylase/reductase cycle, thus inhibiting the post-translational conversion of glutamate residues to gamma-carboxyglutamate. Vitamin K-dependent clotting factors contain essential gamma-carboxyglutamate residues in their Gla domains. Patients receiving coumarin therapy will therefore produce undercarboxylated vitamin K-dependent clotting factors with reduced procoagulant activity. This prolongs the PT time, chiefly due to depression in the levels of factors II, VII and X. Successful oral anticoagulant therapy with coumarins requires careful monitoring of the patient's PT time in order to achieve an effective level of anticoagulation while minimizing bleeding complications (reviewed by Hirsh et al. [14]).

The PT test is accomplished by mixing citrated plasma samples with a thromboplastin reagent and measuring the time to clot formation. The active ingredient in thromboplastin reagents is tissue factor. Before purified TF became available in the 1990s, thromboplastin reagents were made from relatively crude tissue extracts of human or animal origin. More recently, highly purified rTF has been used to prepare thromboplastin reagents that are composed entirely of defined ingredients [15, 16]. Recombinant thromboplastin reagents are potentially superior to tissue-derived reagents because their composition, and therefore their properties, is more readily controlled by the manufacturer. To prepare recombinant thromboplastin reagents, rTF is reconstituted into unilamellar phospholipid vesicles composed of a suitable mixture of phospholipids. (Reconstitution of TF into phospholipid vesicles is sometimes called “relipidation.”) In order to function efficiently in blood coagulation, the vesicles must contain some phospholipids with a net negative charge, with phosphatidylserine being the most effective negatively charged phospholipid. A variety of methods are available for incorporating rTF into phospholipid vesicles (discussed by Smith & Morrissey [17]).

A second pathway for triggering blood clotting is the intrinsic or contact pathway. This tissue factor-independent pathway is activated when plasma comes into contact with certain artificial surfaces, such as glass, silica, or kaolin. The contact pathway is initiated when prekallikrein, high molecular weight kininogen and factor XII are exposed to a negatively charged surface. This results in the formation of an initiator complex that brings about the conversion of factor XII to its active enzyme form, factor XIIa, via limited proteolysis. Factor XIIa then converts factor XI to XIa in a calcium-dependent reaction, which in turn propagates the clotting cascade, leading ultimately to the generation of thrombin and the polymerization of fibrin to create a clot.

In order to measure the levels of all of the hemostatically relevant clotting factors in a patient, it is necessary to perform two clotting tests. One is the PT test, mentioned earlier, and the other is the Activated Partial Thromboplastin Time (aPTT) test. Because the PT test uses tissue factor to activate the clotting cascade, it is sensitive to clotting factors in the extrinsic pathway. The aPTT test uses an artificial activator of clotting (such as kaolin or silica) and is therefore sensitive to changes in the intrinsic pathway. No one test is sensitive to all of the hemostatically relevant clotting factors, so to be certain that a patient does not have a bleeding diathesis (for example, prior to surgery), the clotting ability of the patient's plasma must be evaluated using both tests. In addition, the aPTT is widely used to monitor heparin therapy, and is also the basis for other clinical coagulation assays, such as assays for antiphospholipid antibody syndromes and lupus anticoagulants. The properties of commercial aPTT reagents differ from manufacturer to manufacturer, most particularly with regard to which artificial activator of clotting is used. The aPTT assays have also proven difficult to standardize.

Oligohistidine tags, typically consisting of several consecutive histidine residues incorporated into either the N-terminus or C-terminus of recombinant proteins, are widely used for ease of purification of such proteins [19]. A recombinant fusion protein containing such an oligohistidine tag will bind transition metal ions, such as Ni⁺², with reasonably high affinity. This property can be exploited for affinity purification using derivatives of metal-chelating groups such as nitrilotriacetic acid (NTA) attached to solid supports. NTA will chelate nickel ions, presenting them in such a manner that the bound Ni⁺² can still interact tightly with the oligohistidine tag of recombinant proteins. The recombinant fusion protein bound to immobilized NTA-Ni⁺² complexes can then be specifically eluted with imidazole.

A nickel-chelating lipid, DOGS-NTA-Ni (1,2-dioleoyl-sn-glycero-3-[(N(5-amino-1-carboxypentyl) iminodiacetic acid) succinyl] nickel salt), is commercially available. DOGS-NTA-Ni contains the nickel-binding NTA moiety attached to a dioleoyl-glycerolipid. DOGS-NTA-Ni has chiefly been used by structural biologists to create two-dimensional crystals of oligohistidine-tagged recombinant proteins on artificial membrane surfaces, in order to obtain structural information by electron crystallography [20].

### SUMMARY

In a first aspect, the present invention is a a thromboplastin reagent, comprising: (i) activated sTF, (ii) a metal-chelating lipid, (iii) a metal ion selected from the group consisting of Ni²⁺, Cu²⁺, Co²⁺ and mixtures thereof, and (iv) a phospholipid.

In a second aspect, the present invention is an aPTT reagent, comprising: (i) a metal-chelating agent, (ii) a metal ion selected from the group consisting of Ni²⁺, Cu²⁺, and mixtures thereof, and (iii) a phospholipid.

In a third aspect, the present invention is an activated sTF.

In a fourth aspect, the present invention is a combination PT and aPTT test kit, comprising: (i) activated sTF, (ii) a metal-chelating lipid, (iii) a metal ion selected from the group consisting of Ni²⁺, Cu²⁺, and mixtures thereof, and (iv) a phospholipid.

In a fifth aspect, the present invention is a composition for promoting clotting, comprising: (i) a metal-chelating agent, (ii) a metal ion selected from the group consisting of Ni²⁺, Cu²⁺, Co²⁺, Zn²⁺ and mixtures thereof, and (iii) optionally, activated sTF comprising an extracellular domain of TF and an oligohistidine moiety having at least 2 histidine residues.

Definitions

Ni-NTA-DOGS or DOGS-NTA-Ni means 1,2-dioleoyl-sn-glycero-3-[(N(5-amino-1-carboxypentyl) iminodiacetic acid) succinyl] (Nickel salt).

NTA-DOGS or DOGS-NTA means 1,2-dioleoyl-sn-glycero-3-[(N(5-amino-1-carboxypentyl) iminodiacetic acid) succinyl].

VII or factor VII means coagulation factor VII (zymogen).

VIIa or factor VIIa means coagulation factor VIIa (active enzyme).

X or factor X means coagulation factor X (zymogen).

Xa or factor Xa means coagulation factor Xa (active enzyme).

VIII or factor VIII means coagulation factor VIII (zymogen).

IX or factor IX means coagulation factor IX (zymogen).

XI or factor XI means coagulation factor XI (zymogen).

XIa or factor XIa means coagulation factor XIa (active enzyme).

XII or factor XII means coagulation factor XII (zymogen).

XIIa or factor XIIa means coagulation factor XIIa (active enzyme).

PK means prekallikrein.

HK means high molecular weight kininogen.

PNP means pooled normal plasma.

NTA means nitrilotriacetic acid or a nitrilotriacetic acid moiety.

rTF means recombinant, membrane-anchoring tissue factor.

sTF means soluble tissue factor, a truncated, soluble form of tissue factor that does not have the membrane-spanning domain nor the cytoplasmic domain [3-7].

TF:VIIa means the complex of tissue factor and factor VIIa.

aPTT reagent is a reagent containing an activator of the contact pathway of blood coagulation for use in an aPTT test.

A metal-chelating lipid is a lipid moiety covalently bound to a metal-chelating moiety, such as an NTA-lipid (for example, NTA-DOGS).

A metal-chelating agent contains a covalently bound metal-chelating moiety, such as metal-chelating lipids (for example, NTA-DOGS) and NTA-beads.

His means a histidine moiety or residue.

Oligohistidine is a moiety containing at least two histidine residues. Preferably, the histidine residues are consecutive, in which case the oligohistidine may also be expressed as (His)ₙ, where n is preferably at least 2, more preferably 2-10.

FVII means any protein that exhibits Factor VII clotting activity of human Factor VII. The Factor VII clotting activity of a protein is determined by comparing the amount of the protein necessary to give the same clotting time as human Factor VII in the following assay: 50 μL of citrated Factor VII deficient plasma, together with human Factor VII or the protein, is incubated in a cuvette for 2 min at 37° C., after which clotting is initiated by adding 100 μL pre-warmed thromboplastin reagent, and the time to clot formation is measured with a coagulometer, such as an ST4 coagulometer (Diagnostica Stago, Parsippany, N.J.). The amount of human Factor VII and the type of thromboplastin reagent are preferably selected to give a clotting time of 10-15 seconds. The molar amount of human Factor VII that achieves a given clotting time is divided by the molar amount of the protein that gives the same clotting time, which gives the relative Factor VII clotting activity of the protein. Preferably, FVII has at least 1% of the clotting activity of human Factor VII. FVII includes, for example, natural human Factor VII, natural human Factor VIIa, recombinant human Factor VII [33] and VIIa, and other mammalian Factor VII and VIIa (such as natural rabbit Factor VII and natural rabbit Factor VIIa).

“Factor VIIa equivalents” means that the amount of FVII present has the same clotting activity as the specified amount of natural human Factor VIIa. For example, “10 ng Factor VIIa equivalents of FVII” means that the amount of FVII present has the same clotting activity as 10 ng of natural human Factor VIIa.

Activated sTF means any peptide, protein, or polypeptide which includes a metal binding domain at the C-terminal end (such as (His)ₙ, where n is 2-10), has a solubility that is at least 10% of the solubility of sTF in a 100 mM NaCl solution containing 50 mM Tris-HCl buffer, pH 7.4, and a reagent containing the activated sTF (1 μg/ml), the metal (10 μM) and 15% metal-chelating lipid which chelates the metal, 5% PS, 40% PE and 40% PC (to a total lipid concentration of 100 μM) used as a thromboplastin reagent (100 μL of the reagent pre-warmed to 37° C. is mixed with 50 μL plasma pooled from normal individuals) results in clotting within 1 minute. Examples include sTF(His)₆, sTF-5AA-(His)₆, and sTF-2(His)₅.

A metal binding domain is a moiety which binds a metal in a 100 mM NaCl solution containing 50 mM Tris-HCl buffer, pH 7.4, with at least the affinity of (His)₂. Examples include (His)ₙ, where n is 2-10.

TF means any tissue factor protein, such as rTF and natural mammalian tissue factors.

Thromboplastin reagent is any reagent which contains TF and that when 100 μL of the reagent pre-warmed to 37° C. is mixed with 50 μL plasma pooled from normal individuals will result in clotting within 1 minute; and when neat and warmed to 37° C. does not clot within 2 minutes.

### BRIEF DESCRIPTION OF THE DRAWINGS

FIG. 1 is the amino acid sequences for the C-terminal end of sTF and the three oligohistidine-tagged variants, and the nucleotide sequence encoding those amino acid sequences. Sequences differing from sTF are underlined, with the histidine residues in italics.

FIGS. 2A, B and C are graphs showing the effect of lipid composition on clotting time in a PT assay with 0.3 μg/ml sTF(His)₆ and 100 μM SUV. A: vesicles contain 5% PS, 15% DOGS-NTA-Ni and varying amounts of PE; B: vesicles contain 15% DOGS-NTA-Ni, 40% PE and varying amounts of PS; C: vesicles contain 5% PS, 30% PE and varying amounts of DOGS-NTA-Ni.

FIG. 3 is a graph of the dependence of clot time on total lipid concentration in a PT assay employing 0.3 μg/ml sTF(His)₆ and varying concentrations of 12.5% Ni-lipids.

FIG. 4 is a graph of TF activity in PT assays using modified thromboplastin reagents: rTF/PCPS (circles), sTF/PCPS (open, inverted triangles), sTF(His)₆/10% Ni-lipids (diamonds), and sTF-5AA-(His)₆/10% Ni-lipids (triangles). TF concentration indicated on the x-axis are those in the diluted reagents.

FIG. 5 is a graph of the binding isotherm for the interaction of VIIa with sTF(His)₆ in the presence of 15% Ni-lipids.

FIG. 6 is a bar graph comparison of sTF(His)₆/15% Ni-lipid PT reagent with a commercially-available PT reagent. Clotting times were measured using pooled normal plasma (PNP) or various factor-deficient plasmas.

FIGS. 7A, B and C are graphs showing the effect of lipid composition on clotting time in an aPTT assay with 100 μM SUV. A: vesicles contain 5% PS, 15% DOGS-NTA-Ni and varying amounts of PE; B: vesicles contain 15% DOGS-NTA-Ni, 40% PE and varying amounts of PS; C: vesicles contain 5% PS, 40% PE and varying amounts of DOGS-NTA-Ni.

FIG. 8 is a graph of the dependence of clot time on total phospholipid concentration (15% Ni-lipids) in an aPTT assay.

FIG. 9 is a graph of the effect of varying the preincubation time (prior to adding calcium ions) on clotting time in an aPTT assay using 15% Ni-lipids with pooled normal plasma.

FIG. 10 is a graph of the clotting activity of the 15% Ni-lipids in an aPTT assay on normal pooled plasma (circles) versus VIII-deficient plasma (triangles).

FIG. 11 is a bar graph comparison of 15% Ni-lipid aPTT reagent with commercially-available aPTT reagent. Clotting times were measured in an aPTT assay using pooled normal plasma (PNP) or various factor-deficient plasmas.

FIGS. 12A and B are graphs of the clotting time of PT assays performed with modified thromboplastin reagents containing varying concentrations of NiSO₄ (circles), CuSO₄ (diamonds), CoCl₂ (triangles), or ZnCl₂ (open, inverted triangles). The metal ion concentrations indicated on the x-axis are the concentrations employed in the modified reagent. A and B are plots of the same data, over different ranges of metal ion concentrations.

FIG. 12C is a graph of the clotting time of aPTT assays performed with modified reagents containing varying concentrations of NiSO₄ (circles), CuSO₄ (diamonds), CoCl₂ (triangles), or ZnCl₂ (open, inverted triangles). The metal ion concentrations indicated on the x-axis are the concentrations employed in the modified reagent.

FIG. 13 is a graph of clotting times of sTF(His)₆ (squares), sTF-2(His)₅ (diamonds), and sTF-5AA-(His)₆ (inverted triangles) in a PT assay. Each type of TF was incubated with SUV containing increasing proportions of DOGS-NTA-Ni.

FIG. 14 is a graph of binding isotherms for the interaction of VIIa with different types of TF in the presence of 15% Ni-lipid: sTF(His)₆ (squares), sTF-2(His)₅ (diamonds), and sTF-5AA-(His)₆ (inverted triangles).

FIG. 15 is a schematic of the clotting cascades.

FIGS. 16 (a) and (b) are graphs showing EC₅₀ for the ability of sTF-5AA-(His)₆ to enhance the rate of X activation by VIIa. Varying concentrations of purified sTF or sTF-5AA-(His)₆ were incubated in wells of 96-well plates that had previously been coated with a mixture of either 10% DOGS-NTA-Ni, 20% PS, 70% PC (closed symbols) or 20% PS, 80% PC (open symbols); initial rates of Xa generation were measured using 40 nM X and are expressed as percent of the activity observed with 1 μM sTF-5AA-(His)₆ in wells coated with lipid mixtures containing DOGS-NTA-Ni. (a) Concentration dependence of the ability of sTF-5AA-(His)₆ (circles) or sTF (squares) to enhance the rate of X activation by VIIa. (b) Data for sTF-5AA-(His)₆ from panel (a) are plotted with the x-axis expanded from 0 to 50 nM.

FIG. 17 is a graph of clotting activity of sTF-5AA-(His)₆ in conjunction with lipid mixtures that were dried in the wells of polystyrene coagulometer cuvettes. The amount of lipid dried per well is given in the x-axis. Each well also received a 50 μl aliquot of 20 nM sTF-5AA-(His)₆. Clotting times were determined using pooled normal human plasma. Lipid mixtures were: NiPCPSPE (solid circles); PCPS (open inverted triangle); PCPSPE (open square); and NiPCPS* (open diamond).

FIG. 18 is a graph of clotting activity of sTF-5AA-(His)₆ in coagulometer cuvettes coated with 200 nmol NiPCPSPE per well. Increasing concentrations of sTF-5AA-(His)₆ (solid circles) or a single concentration of sTF (4800 nM; open inverted triangle) were incubated with the wells prior to adding plasma and calcium ions to initiate clotting. The concentrations indicated on the x-axis refer to the concentration of sTF or sTF-5AA-(His)₆ in the 50 μl aliquot added to each well.

### DETAILED DESCRIPTION

Typically, recombinant TF must contain the transmembrane domain or an equivalent membrane anchoring domain in order to express full procoagulant activity and therefore be suitable for use in recombinant thromboplastin reagents. Compared to sTF, however, rTF is expressed at much lower levels, is more difficult to purify, and is more difficult to handle. In addition, the reconstitution of rTF into phospholipid vesicles is laborious. We therefore sought to develop a form of recombinant TF that would have all of the desirable expression, handling, and solubility characteristics of sTF, but which would exhibit procoagulant activities in plasma clotting tests that were comparable to relipidated rTF.

Attaching a metal binding domain, such as an oligohistidine tag, to the C-terminus of sTF allows the protein to bind to phospholipid vesicles that contain metal-chelating lipid. Since the C-terminal portion of the extracellular domain of wild-type TF connects to the transmembrane domain via a short peptide sequence [18], attaching the oligohistidine tag to the C-terminus of sTF (optionally, with spacers such as other amino acid residues) allows it to orient properly when bound to phospholipid surfaces via a metal chelated to metal-chelating lipids.

A metal chelated to a metal-chelating lipid, such as DOGS-NTA-Ni, can be readily incorporated into phospholipid bilayers, allowing the metal to bind a metal binding domain of recombinant proteins, for example an activated sTF. Furthermore, it has also been discovered that activated sTF bound to vesicles in this manner behaves substantially like membrane-anchoring rTF and has procoagulant activities that are comparable to rTF.

It has also been discovered that membrane bilayers containing nickel-chelating lipids that have been immobilized onto a solid support can efficiently capture activated sTF from crude mixtures, simultaneously purifying the protein and anchoring it to the membrane in one simple step. Furthermore, the ability to bind highly active preparations of activated sTF onto immobilized phospholipid bilayers containing metal-chelating lipids can be used to prepare point-of-care clinical coagulation assays in which the activator of clotting is attached to a chip surface.

A highly active PT reagent (thromboplastin reagent) can be prepared using activated sTF, such as sTF(His)₆ or sTF-5AA-(His)₆, in the presence of phospholipid vesicles (for example, containing 10% DOGS-NTA-Ni, 5% PS, 30% PE, and 55% PC).

In addition to creating a potent thromboplastin reagent, it has also been discovered that even in the absence of any tissue factor, metal-chelating lipids with Ni²⁺ or Cu²⁺, such as DOGS-NTA-Ni, in phospholipid vesicles are also very strong activators of the contact pathway of blood clotting. These phospholipid vesicles may be used as both diagnostic and therapeutic agents. They can serve as the active ingredient in a chemically-defined aPTT reagent. They may also be used in treating bleeding episodes in patients.

A PT reagent (thromboplastin reagent) can be prepared from activated sTF in the presence of a metal-chelating lipid that has bound metal ions, preferably transition metal ions, such as Ni²⁺, Cu²⁺, Zn²⁺ or Co²⁺, with Ni²⁺ and Cu²⁺ being the most potent. A highly active aPTT reagent can be prepared from a metal-chelating agent that has bound metal ions, preferably transition metal ions, such as Ni²⁺, Cu²⁺, Zn²⁺ or Co²⁺, with Ni²⁺ being the most potent.

Metal-chelating agents include NTA beads and metal-chelating lipids. Examples of metal-chelating lipids include: 1-palmitoyl-2-[8-[(E,E)-2′,4′-hexadienoyloxy]octanoyl]-sn-glycero-3-N-[11-[N′,N′-bis[carboxymethyl]imino]-3,6,9-trioxaundecanoyl] phosphatidylethanolamine (which chelates, for example, Cu through an iminodiacetate (IDA) moiety) [35], lipid distearyl imino-diacetate (DSIDA) (which chelates, for example, Cu) [36], and 1,2-Dioleoyl-sn-Glycero-3-[(N-(5-amino-1-carboxypentyl)iminodiacetic acid)succinyl](Ammonium Salt)(DOGS-NTA) (which chelates, for example, Ni).

Activated sTF preferably includes the extracellular domain of TF and an oligohistidine moiety having at least 2 histidine residues, more preferably 2-10 histidine residues. Preferably, the histidine residues are consecutive. More preferably, activated sTF includes (His)ₙ, where n is 2-10, more preferably 4-6. Examples of activated sTF include sTF(His)₆, sTF-5AA-(His)₆, and sTF-2(His)₅.

PT test kit includes the thromboplastin reagent. An aPTT test kit includes an aPTT reagent. Optionally, a Ca²⁺ containing reagent, a buffer, and/or a preservative may be included in either kit. Similarly, a combination kit for both a PT test and an aPTT test would include a reagent which may be formed into either a thromboplastin reagent of an aPTT reagent, for example Ni²⁺ and/or Cu²⁺ and a metal-chelating lipid, with the activated sTF packaged separately.

Examples of clotting assays for either the extrinsic pathway (PT) or intrinsic pathway (aPTT) include: A PT assay may contain oligohistidine-tagged sTF and either Ni²⁺ or Cu²⁺ in combination with NTA-lipids; an aPTT assay may contain Ni²⁺ in combination with NTA-lipids.

An example of a formulation that potently activates both the extrinsic and intrinsic coagulation pathways simultaneously (for example, as a therapeutic), can be prepared using oligohistidine-tagged sTF in combination with Ni²⁺ or Cu²⁺ and NTA-lipids.

An example of a kit for a combination PT test and aPTT test includes three bottles of reagent, listed below as Reagents A, B and C, followed in each case by a listing of their contents. The bottles may contain these ingredients as ready-made solutions, or they can be lyophilized. In the latter case, the reagents may be reconstituted by adding an appropriate volume of water to yield the indicated final concentrations of constituents.

Reagent A

(a) 100 μM Ni-lipids

(b) an optional suitable buffer (for example, 25 mM Tris-HCl buffer, pH 7.4)

(c) an optional preservative (for example, 0.1% w/v NaN₃)

Reagent B

(d) 1 to 100 nM activated sTF

(e) optionally, 25 mM CaCl₂

(f) an optional suitable buffer (for example, 25 mM Tris-HCl buffer, pH 7.4)

(g) an optional stabilizer (e.g., 0.1% w/v bovine serum albumin)

(h) an optional preservative (e.g., 0.1% w/v NaN₃)

Reagent C (Optional)

(i) 25 mM CaCl₂ in water

(j) an optional preservative (e.g., 0.1% w/v NaN₃)

PT Assay (Semi-Automated)

1. Mix equal volumes of Reagents A and B together to create the Thromboplastin Reagent.

2. Pipet 50 μl citrated plasma into a coagulometer cuvette.

3. Incubate 120 sec. to ensure the plasma has reached 37° C.

4. Add 100 μl Thromboplastin Reagent, mix, and measure the time to clot formation from the point of addition of the Thromboplastin Reagent.

Alternative PT Assay (Fully Automated)

1. Pipet 50 μl citrated plasma into a coagulometer cuvette.

2. Incubate 120 sec. to ensure the plasma has reached 37° C.

3. Add 50 μl Reagent A followed immediately by 50 μl Reagent B. Mix. Measure the time to clot formation from the point of addition of Reagent B.

This version of the assay could be appropriate for automated coagulometers, in which the lag between the addition of Reagents A and B in step 3 would be as short as possible.

aPTT Assay

1. Pipet 50 μl citrated plasma into a coagulometer cuvette.

2. Add 50 μl Reagent A. Mix.

3. Incubate 180 sec. at 37° C.

4. Add 50 μl Reagent C, mix, and measure the time to clot formation from the point of addition of Reagent C.

Therapeutic compositions may be formed, to stop bleeding from a wound, by contacting blood from the wound, or by contacting the wound with the composition. The composition may contain the components needed to initiate the intrinsic pathway, the extrinsic pathway, or both. The components may be the same as those used in the thromboplastin reagent, the aPTT reagent, or both. The therapeutic composition may be in a variety of forms, depending on the location of the wound: a topical composition, a nasal spray, a suppository, a mouthwash, an injectable composition, a bandage and a wound dressing. Therapeutic compositions are preferably sterile, and may contain preservatives. Therapeutic compositions may be administered in a wide variety of forms including unit dosage forms, and may be combined with various pharmaceutically acceptable carriers. Such carriers include solid diluents or fillers, sterile aqueous media and various non-toxic organic solvents. Moreover, oral compositions (including mouthwash, and pads or swabs impregnated with therapeutic compositions) can be suitably sweetened and/or flavored.

Bandage and wound dressing may contain the composition in wet or dry (lyophilized) form. A nasal spray may contain the composition in wet or dry powder form, together with other customary additives and/or carriers, such as those described in U.S. Pat. No. 6,815,424.

A mouthwash will contain the composition in wet form, optionally contain other ingredients common to a mouthwash. Examples include those described in U.S. Pat. No. 5,945,087 and U.S. Pat. No. 5,338,538.

An injectable form may include a pharmaceutically acceptable carrier. An injectable composition may be injected into any body cavity, but typically not intravenously.

A topical composition may be in wet or dry powder form, and may include a topically acceptable carrier. Examples of such topically acceptable carriers may be found in International Patent Publication WO 00/62742, published Oct. 26, 2000; U.S. Pat. Nos. 5,691,380; 5,968,528; 4,139,619; and 4,684,635. Suitable topically acceptable carriers, as well as other pharmaceutical carriers, are also described in Remington's Pharmaceutical Sciences, 17th ed., Mack Publishing Company, Easton, Pa. (1990), which is a standard reference text in this field.

Preferably, the thromboplastin reagent contains Ca²⁺, or the Ca²⁺ may be added just prior to use of the reagent. The Ca²⁺ may be provided with the thromboplastin reagent in a kit, with each part separately packed, optionally with each reagent in dry form. Ca²⁺ is preferably added as CaCl₂. The amount of Ca²⁺ is preferably 1-100 mM, more preferably 5-75 mM, more preferably 15-50 mM, including 20 mM, 25 mM, 30 mM, 35 mM, 40 mM and 45 mM.

Ionic strength may be adjusted by adding salts, such as alkali metal and alkaline earth metal salts, including halides, sulfates, nitrates and acetates, such as NaCl and KCl. Preferably, the salts are present in an amount of 0-200 mM, 10-150 mM, 15-125 mM, or more preferably 25-100 mM.

Preferably, the thromboplastin reagent does not contain one or more of Factor II, Factor X, actin, hexokinase, and alkaline phosphatase. The absence of actin, hexokinase and alkaline phosphatase indicates that the thromboplastin reagent does not contain tissue extracts (although the tissue factor itself may have been isolated and purified from tissue).

The thromboplastin reagents contain TF relipidated into phospholipids, such as phosphatidylcholine (PC), phosphatidylserine (PS) and phosphatidylethanolamine (PE). At least a portion of the phospholipids are net negatively charged phospholipids, such as PS, phosphatidylglycerol (PG), phosphatidic acid (PA), and phosphatidylinositol (PI). Preferably, the amount of PS is from 5-50%, more preferably from 10-40%, including 15%, 20%, 25%, 30%, and 35%, of the total phospholipids content. The amount of PE is preferably 0-50%, more preferably 5-40%, including 10%, 15%, 20%, 25%, 30%, and 35%, of the total phospholipids content. Preferably, the remainder of the phospholipids content is composed of neutral phospholipids, such as PC, for example 0-95%, more preferably 40-90%, including 45%, 50%, 55%, 60%, 65%, 70%, 75%, 80%, and 85%, of the total phospholipid content.

The International Sensitivity Index (ISI) value of the thromboplastin reagent is preferably 0.6 to 2, more preferably 0.8 to 1.5, even more preferably 0.8 to 1.2, and most preferably 0.9 to 1.1. Alternatively, preferably the ISI value of the thromboplastin reagent is at most 1.5 or at most 1.2. ISI value of the thromboplastin reagent should be determined by the WHO approved method [34].

The thromboplastin reagents may be provided in dried form, by freeze-drying, spray-drying, or other suitable protein drying methods. The reagents may be dried onto strips or other solid supports, and may be provided as kits with the components provided separately packaged, or groups of the components packaged into 2 or more packages. Some or all of the components may be provided in dried forms, and other components provided in saline or a physiological buffer.

Thromboplastin reagents of the present invention may contain added FVII. Addition of minute amounts of Factor Vila to the thromboplastin reagent can be used to minimize sensitivity to Factor VII and to further manipulate responses to other factors. This is more fully explained in “THROMBOPLASTIN REAGENTS” U.S. patent application Ser. No. 10/931,282 to Morrissey et al., filed on Aug. 31, 2004. Any FVII may be used including any mammalian Factor VII or VIIa (such as human, rabbit, rat, cow, etc.). Preferably the thromboplastin reagents contain added Factor VIIa, more preferably human Factor VIIa. The FVII may be prepared recombinantly

Preferably, the amount of FVII present is less than the amount of Factor VII or Factor VIIa found in the plasma of normal individuals, including the amount of Factor VII or VIIa found in Factor II- and Factor X-deficient plasmas. The amount of FVII present is preferably 0.1 to 10 nanograms/milliliter (ng/ml) Factor VIIa equivalents, 1 to 6 ng/ml Factor VIIa equivalents, or 2.5 to 5 ng/ml Factor Vila equivalents, and more preferably at least 1 ng/ml or at least 2.5 ng/ml Factor VIIa equivalents. Alternatively, the amount of FVII may be expressed in picomolar (pM) amounts; such as 1-1000 pM Factor VIIa equivalents, 50-400 pM Factor VIIa equivalents, preferably at least 150 pM Factor VIIa equivalents or at least 200 pM Factor VIIa equivalents.

Thromboplastin reagents may be used to monitor any anticoagulant drug therapy. Table A below lists a variety of these drugs.

TABLE A
Anticoagulant drugs that may be monitored with thromboplastin reagents

| | |
|---|---|
| Coumarin Derivatives | Warfarin (COUMADIN ®)¹ |
| (block production of | Nicoumalone (ACENOCOUMAROL ™)¹ |
| functional factors II, VII, | Dicoumarol (BISHYDROXYCOUMARIN ™) |
| and X): | Phenprocoumon |
| Thrombin | Argatroban (NOVASTAN ®)¹ |
| (FIIa)Inhibitors | Ximelgatran (EXANTA ®)² |
| | BIBR 1048² |
| | BIBR 953 |
| | Desirudin (REVASC ®)¹ |
| | Lepirudin (REFLUDAN ® or |
| | PHARMION ®)¹Bivalirudin (ANGIOMAX ®, |
| | previously HIRULOG ®)¹ |
| FXa Inhibitors | DX-9065a² |
| | DPC 906² |
| | Antistasin³ |
| TF/FVIIa Inhibitors | Anti-TF antibodies |
| | Recombinant Nematode Anticoagulant Protein |
| | (rNAPc2)² |
| | Recombinant Tissue Factor Pathway Inhibitor |
| | (TIFACOGIN ™)² |
| | FVIIai³ |

ART-123 ™ (recombinant soluble thrombomodulin)²
¹FDA approved for use in humans
²Evaluated in clinical trials but not yet approved
³Still in development (animal studies only)

### EXAMPLES

Unless otherwise stated, the following studies were performed using sTF(His)₆ and employed SUVs prepared by the Bio-Bead method.

Phospholipids for the Modified Thromboplastin Reagents

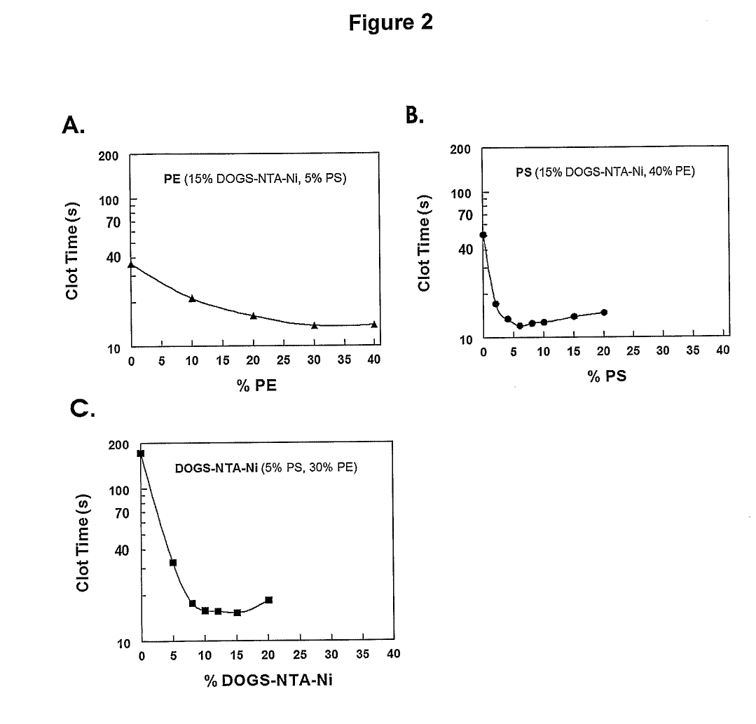

The procoagulant properties of sTF(His)₆ were first studied by determining the composition of phospholipids that produced the shortest clot times in a PT assay. The proportion of PE, PS, PC, and DOGS-NTA-Ni in SUVs was systematically altered and their effectiveness in supporting the clotting of normal pooled plasma was tested. Mixtures of SUVs (100 μM lipid) and 0.3 μg/ml sTF(His)₆ were used to create modified thromboplastin reagents for the PT clotting assay. The shortest clotting times with these reagents were obtained when the SUVs contained 12% Ni-lipid (40% PE, 5% PS, 12% DOGS-NTA-Ni, and 43% PC) (FIG. 2).

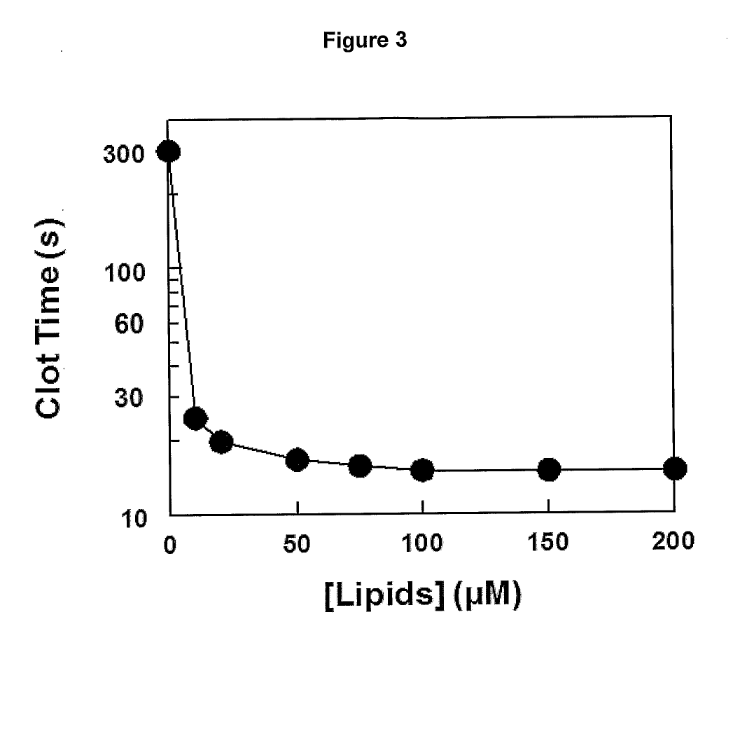

Also examined were SUV concentrations that resulted in the shortest clotting times. Using the PT assay and 0.3 μg/ml sTF(His)₆ we varied the concentration of total phospholipids containing 12.5% DOGS-NTA-Ni. Clotting times became shorter as the SUV concentration increased, reaching a plateau at approximately 100 μM phospholipid (FIG. 3).

Properties of Oligohistidine-Tagged sTF

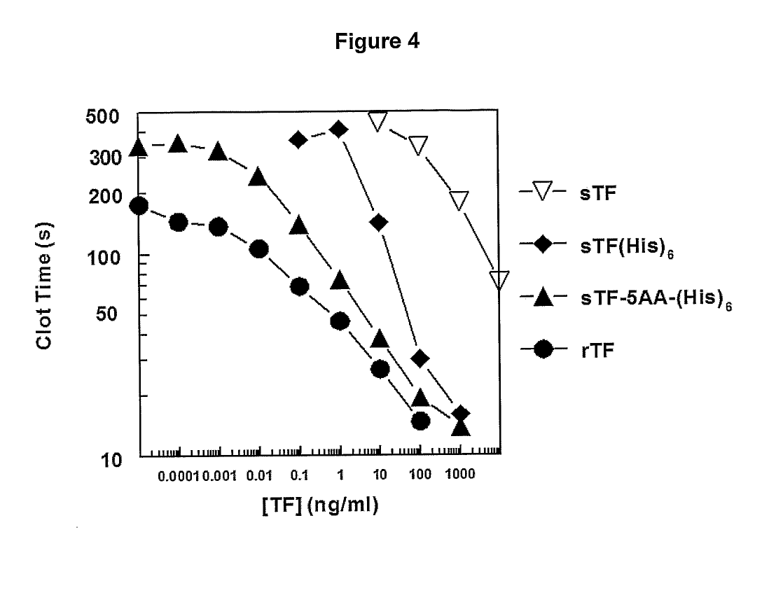

The clotting activities of sTF(His)₆ and sTF-5AA-(His)₆, in the presence of phospholipid vesicles containing 10% DOGS-NTA-Ni, 5% PS, 30% PE and 55% PC (referred to as 10% Ni-lipids), were compared to the activity of sTF in the presence of PCPS vesicles, and rTF relipidated into PCPS vesicles. Thromboplastin reagents containing TF and phospholipids were prepared and diluted to various concentrations of TF. The diluted thromboplastin reagents were then used in the PT assay (FIG. 4). The four modified thromboplastin reagents were made as follows: 1. 100 ng/ml rTF relipidated in PCPS vesicles (30 μM), 2. 1000 ng/ml sTF(His)₆ plus 10% Ni-lipids (100 μM), 3. 1000 ng/ml sTF-5AA-(His)₆ plus 10% Ni-lipids (100 μM), and 4. 10,000 ng/ml sTF plus PCPS vesicles (100 μM). The thromboplastin reagents were diluted in TA (50 mM Tris-HCl buffer, pH 7.5, 0.1% bovine serum albumin, 0.1% NaN₃) to varying concentrations of TF.

The TF concentration that yielded a 50 sec. clot time was used to compare the activities of the different thromboplastin preparations (Table 1). The procoagulant activities of both sTF(His)₆ and sTF-5AA-(His)₆ were dramatically higher than sTF. sTF-5AA-(His)₆ was the more active variant, with procoagulant activity within a factor of ten of that of rTF.

The ability of sTF(His)₆ to allosterically activate VIIa was compared to sTF and rTF/PCPS. sTF(His)₆ was also able to fully allosterically activate VIIa (data not shown).

TABLE 1
Clotting activity of various forms of recombinant
tissue factor in the PT assay

| | | Concentration of TF that | |
|---|---|---|---|
| TF | Phospholipid | yields a 50 sec. clot time** | |
| sTF | PCPS | >100 | μg/ml |
| sTF-(His)₆ | Ni-NTA- | 45 | ng/ml |
| | DOGS/PCPSPE* | | |
| sTF-5AA- | Ni-NTA- | 4.8 | ng/ml |
| (His)₆ | DOGS/PCPSPE* | | |
| rTF | PCPS | 0.7 | ng/ml |

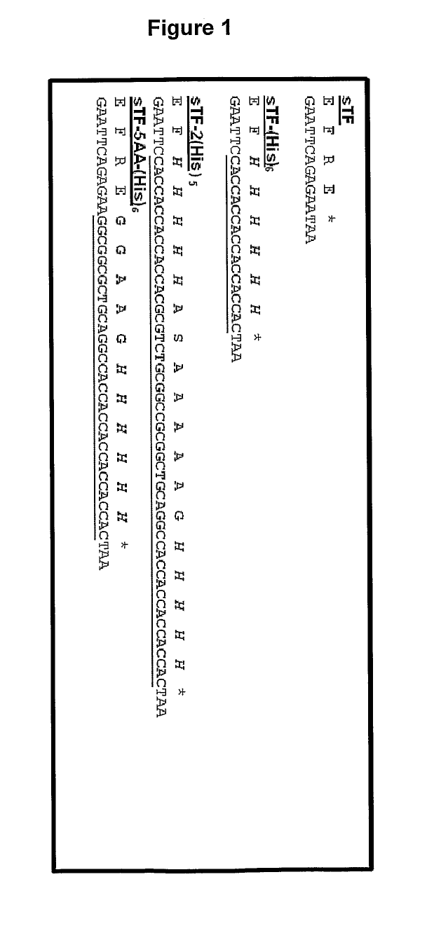

*SUV with 10 mol % Ni-NTA-DOGS
**Clotting data are from FIG. 1

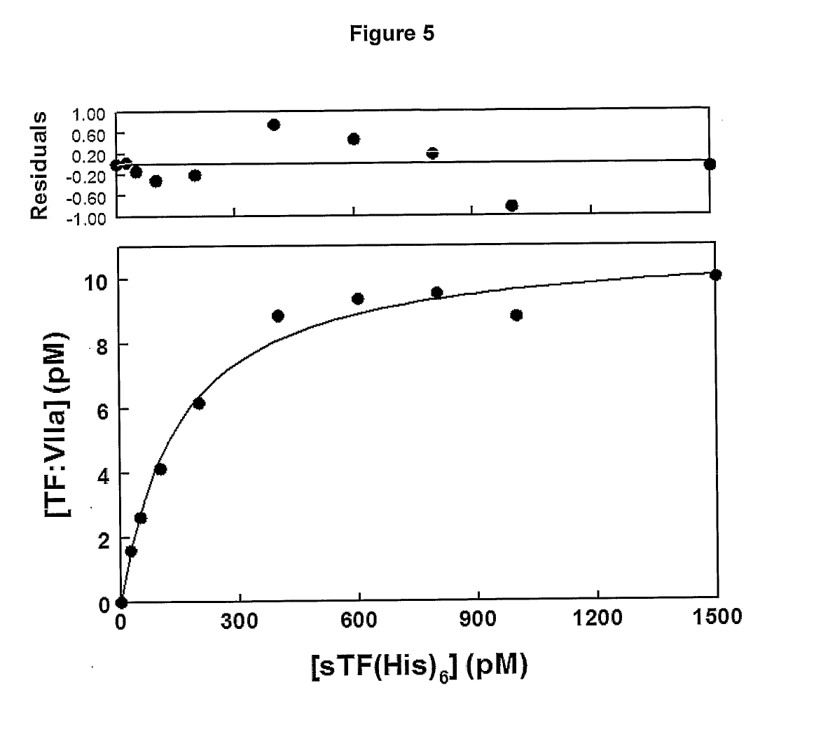

sTF-(His)₆ was examined for how well it binds to VIIa. Membrane-anchoring rTF and sTF differ dramatically in their binding affinities for VIIa. VIIa binds extremely tightly to rTF that has been relipidated into PCPS vesicles, with K_{d} values that are less than 50 pM [4]. On the other hand, VIIa binds considerably more weakly to sTF, with K_{d} values of approximately 2 to 5 nM [4]. The K_{d} values were determined for the binding of Vila to rTF/PCPS, to sTF in the presence of PCPS vesicles, and to sTF(His)₆ in the presence of Ni-lipids (Table 2; see FIG. 5 for a typical binding isotherm for the binding of VIIa to sTF(His)₆ in the presence of Ni-lipids.)

TABLE 2
Dissociation constants for VIIa binding to various
forms of recombinant human tissue factor

| | | K_{d.app} |
|---|---|---|
| TF | Phospholipid | pM ± SEM |
| sTF | PCPS | 7 (±3) × 10³ |
| sTF-(His)₆ | Ni-NTA-DOGS/PCPSPE* | 169 ± 15 |
| rTF | PCPS | 40 ± 5 |

*SUV with 15 mol % Ni-NTA-DOGS

Consistent with literature values, a K_{d} value was obtained for VIIa binding to rTF/PCPS of 40 μM, and a K_{d} value was obtained for VIIa binding to sTF of 7 nM. This confirms previous reports that VIIa binds to rTF/PCPS with an affinity that is approximately 100-fold higher than the affinity with which it binds to sTF. Interestingly, it was found that VIIa bound with very high affinity to sTF(His)₆ in the presence of Ni-lipids.

The experiments mentioned thus far in this study were performed with phospholipid vesicles made via the Bio-Bead method. Similar clotting experiments were performed with vesicles made either by sonication or extrusion, with similar results (data not shown). Thus, all three types of vesicles supported the clotting activity of sTF(His)₆. Vesicles prepared by the Bio-Bead method supported the highest activity (shortest clot times) compared to extruded or sonicated vesicles.

Oligohistidine-Tagged sTF and Ni-Lipids as a PT Reagent

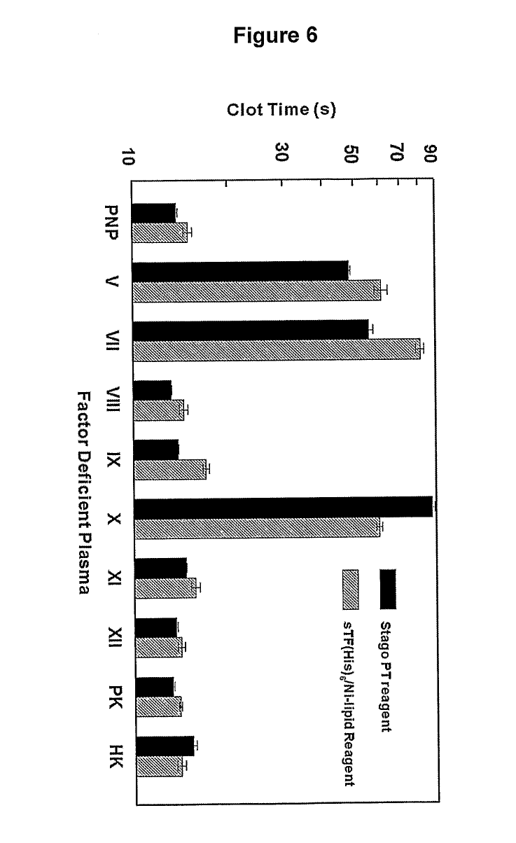

A major goal was to create a soluble form of TF that can function as a thromboplastin reagent in the PT assays. To test this, a modified thromboplastin reagent was created containing the following final concentrations: 3 μg/ml sTF(His)₆, and 100 μM 15% Ni-lipid. This reagent was then compared in PT assays to a commercially available thromboplastin reagent, STA-Neoplastine CI Plus (Diagnostica Stago). Clotting times with the two reagents were compared using pooled normal plasma as well as a number of factor-deficient plasmas (FIG. 6). (The latter were deficient in factors V, VII, VIII, IX, X, XI, XII, prekallikrein (PK), or high molecular weight kininogen (HK)). As expected for PT assays, both reagents exhibited prolonged clotting times with plasmas deficient in factors V, VII or X, while being insensitive to deficiencies in clotting factors of the intrinsic pathway (factors VIII, IX, XI, XII, PK, or HK).

Phospholipids for the aPTT Assay

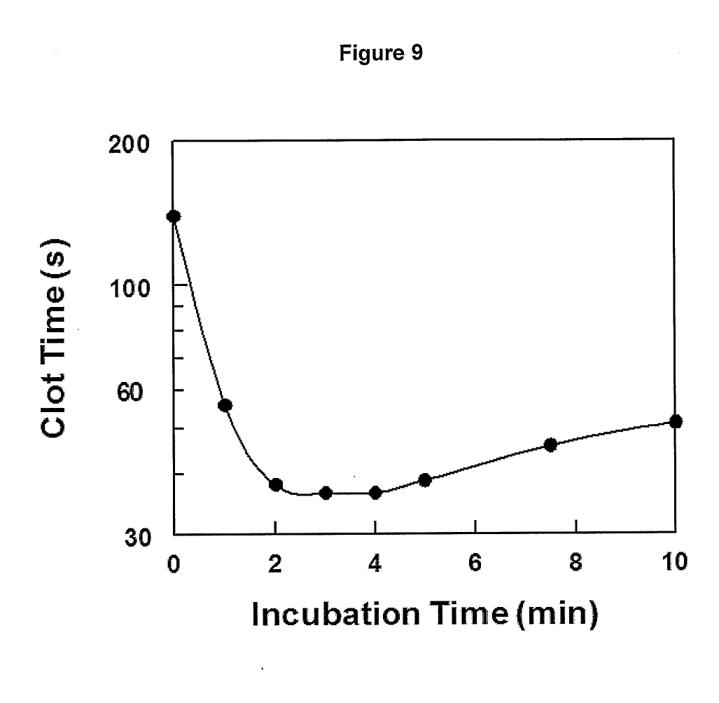

It was discovered that, under some circumstances, Ni-lipids are themselves procoagulant, even in the absence of sTF(His)₆. Further studies indicated that Ni-lipids are potent activators of the contact pathway of blood clotting, particularly when preincubated with plasma at 37° C. for 2 to 4 min prior to the addition of calcium ions (see FIG. 9). (In the PT test data presented above, the Ni-lipids were not preincubated with plasma, and so the contact pathway of blood clotting was not activated to any significant extent.)

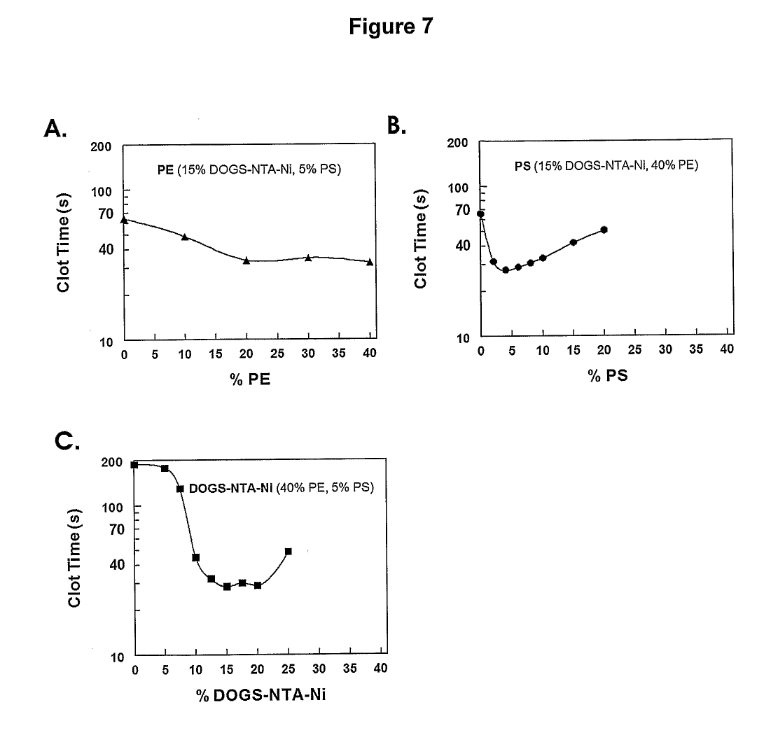

The ability of Ni-lipids to activate the contact pathway was explored in the following series of experiments. In the first experiments, the phospholipid dependence of Ni-lipid procoagulant activity was examined by varying the contents of PS, PE, PC and DOGS-NTA-Ni in an aPTT assay (FIG. 7). The shortest clotting times were obtained with SUVs composed of 15% DOGS-NTA-Ni, 5% PS, 40% PE and 40% PC (referred to as 15% Ni-lipids).

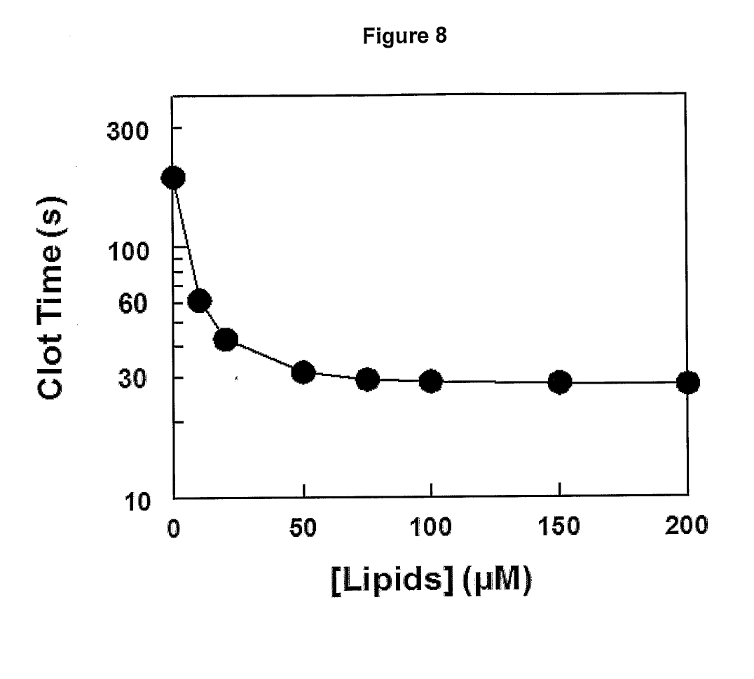

Next, the influence of phospholipid concentration on clotting time was examined in the aPTT assay using 15% Ni-lipids (FIG. 8). The shortest clotting times were obtained at phospholipid concentrations at or above 100 μM.

The time of preincubation of Ni-lipids with plasma (before adding calcium ions) was important for the performance of the aPTT assay (FIG. 9). In the absence of preincubation, clotting times were very long (>100 sec). The optimal duration of preincubation of Ni-lipids with plasma was 2 to 4 min, with clotting times becoming longer thereafter. This behavior is typical of aPTT reagents and is comparable to commercially-available aPTT assays.

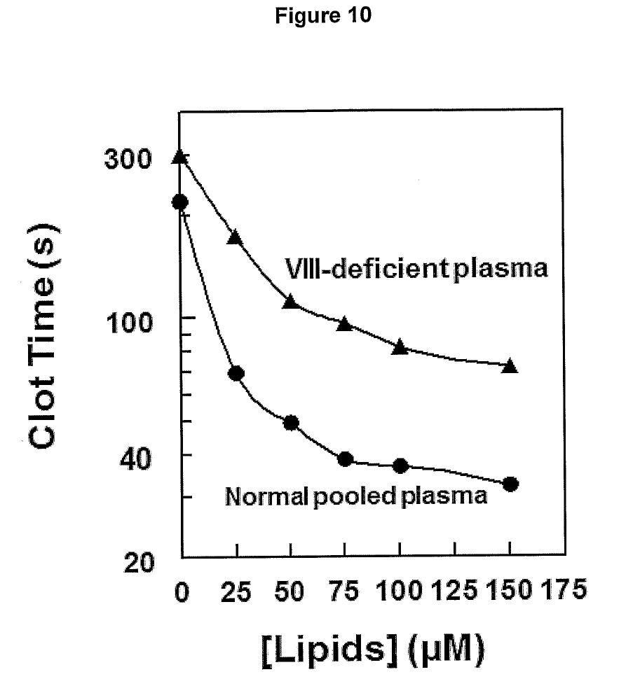

In order to establish that the procoagulant activity of Ni-lipids was due to activation of the contact pathway, the Ni-lipid-based aPTT assay was repeated with normal versus factor VIII-deficient plasma (FIG. 10). Normal plasma clotting times shortened dramatically with increasing concentrations of 15% Ni-lipids, while the clotting times with factor VIII-deficient plasma were significantly prolonged in these assays at all vesicle concentrations tested. This demonstrates that the procoagulant activity of Ni-lipids depends upon the intrinsic pathway of blood clotting.

Ni-Lipids as an aPTT Reagent

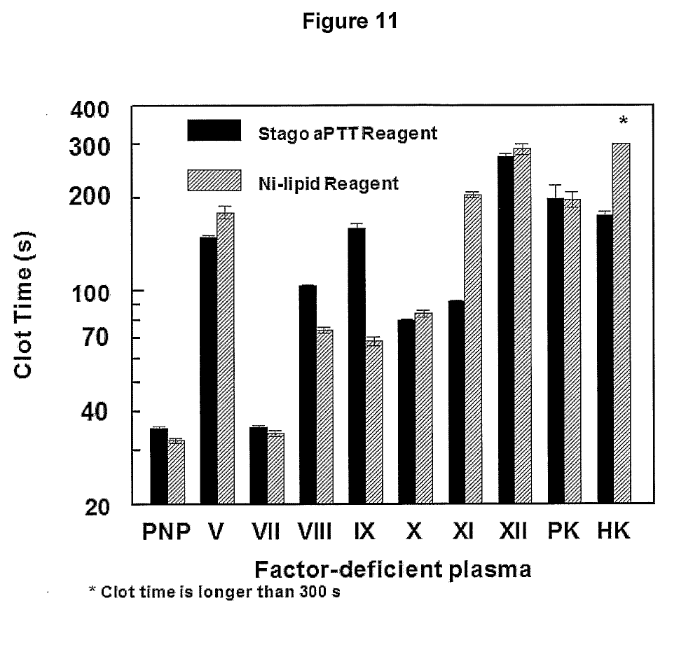

The procoagulant activity of Ni-lipids was tested in aPTT assays in comparison to a commercially-available aPTT reagent using normal pooled plasma and plasmas deficient in various individual clotting factors (FIG. 11). For the Ni-lipid reagent, 50 μM phospholipid vesicles containing 15% DOGS-NTA-Ni, 5% PS, 40% PE and 40% PC was used. The commercially-available aPTT reagent was STA-PTT-Automate 5 (Diagnostica Stago). A concentration of 50 μM Ni-lipid was chosen because it yielded a baseline aPTT clotting with normal pooled plasma that was similar to the clotting time of the commercial aPTT reagent with normal pooled plasma. The aPTT assays were performed by incubating 50 μl of aPTT reagent with 50 μl plasma for 3 minutes at 37° C., then initiating clotting by adding 50 μl of pre-warmed 25 mM CaCl₂ (FIG. 11). As expected, clotting times for both reagents were insensitive to deficiency in factor VII, which is specific for the extrinsic pathway of blood clotting. Also as expected, clotting times with both reagents were prolonged when plasmas were deficient in any of the following clotting factors: factor V, VIII, IX, X, XI, XII, prekallikrein, or high molecular weight kininogen. This confirms that Ni-lipids activate the coagulation cascade through the contact pathway of blood clotting. These results also demonstrate that aPTT reagents based on Ni-lipids have properties that are highly comparable to those of commercially-available aPTT reagents.

Metal Ion Specificity of the Ni-Lipids as Contact Activators

In this series of experiments, the ability of a variety of other transition metals to support the novel PT and aPTT assays when bound to NTA-DOGS was tested. These experiments were performed by preparing mixed phospholipid vesicles (using the Bio-Bead method) containing the following mol % lipids: 15% NTA-DOGS, 5% PS, 40% PE, 40% PC. These mixed phospholipid vesicles (termed NTA-lipids here) were then used in the aPTT and PT assays.

The modified thromboplastin reagent contained varying concentrations of the indicated metal salts, NTA-lipids (100 μM lipid), 30 ng/ml sTF-5AA-(His)₆, 0.004% (w/v) bovine serum albumin, 0.08% (w/v) sodium azide, and 16 mM Hepes buffer, pH 7.4. The modified aPTT reagent contained varying concentrations of the indicated metal salts, NTA-lipids (100 μM lipid), 0.08% (w/v) sodium azide, and 16 mM Hepes buffer, pH 7.4.

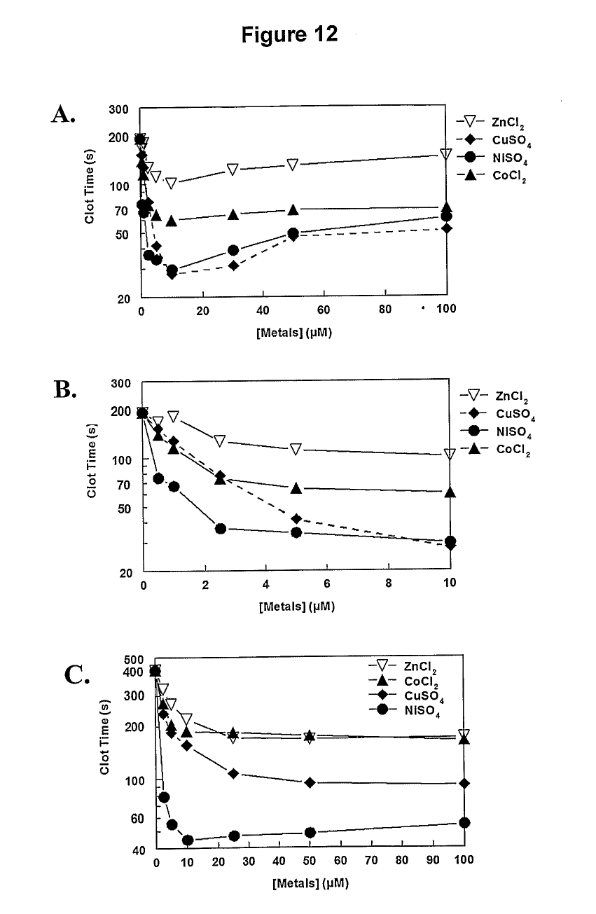

PT assays were performed with modified thromboplastin reagents containing 30 ng/ml sTF-5AA-(His)₆ and varying concentrations of NiSO₄, CoCl₂, CuSO₄, ZnCl₂, FeSO₄, CdCl₂, CrCl₂, AgNO₃ or MnCl₂ in the presence of NTA-lipids (FIG. 12). (A low concentration of sTF-5AA-(His)₆ (30 ng/ml) was chosen for this set of experiments, so that an easily observable range of clotting times could be obtained as the concentrations of metal ions was varied. In a more typical PT assay, higher concentrations of sTF-5AA-(His)₆ should be used, yielding clotting times with normal pooled plasma in the range of 10 to 15 sec.)

Several of the metal ions tested (Fe²⁺, Cd²⁺, Cr²⁺, Ag⁺, and Mn²⁺) at concentrations ranging from 0 to 90 μM exhibited prolonged clotting times (>200 sec) in this PT assay when included in the modified thromboplastin reagent (data not shown). Zn²⁺ showed some activity (see FIG. 12). In contrast, Ni²⁺, Cu²⁺ and Co²⁺ all dramatically shortened the clotting times in a concentration-dependent manner when added to the modified thromboplastin reagent (FIG. 12). The shortest clotting times (<30 sec) were obtained with 10 μM CuSO₄ or NiSO₄, indicating that Cu²⁺ and Ni²⁺ have comparable activities in this assay system. At metal ion concentrations less than 10 μM, however, Ni²⁺ supported shorter clotting times than did Cu²⁺ (FIG. 12B). Co²⁺ was the only other metal tested that was able to shorten the PT clotting time to less than 100 sec when added to the modified thromboplastin reagent. However, none of the Co²⁺ concentrations tested supported clotting times as short as those observed with optimal concentrations of Cu²⁺ or Ni²⁺.

aPTT assays were performed with modified aPTT reagents containing varying concentrations of NiSO₄, CoCl₂, CuSO₄, ZnCl₂, FeSO₄, CdCl₂, CrCl₂, AgNO₃ or MnCl₂ in the presence of NTA-lipids (FIG. 12C). Several of the metal ions tested (Fe²⁺, Cd²⁺, Cr²⁺, Ag⁺, and Mn²⁺) all exhibited prolonged clotting times (>200 sec) in the aPTT assay when added to the modified aPTT reagent at concentrations ranging from 0 to 90 μM (data not shown). Co²⁺ and Zn²⁺ some activity (see FIG. 12C). In contrast, both Ni²⁺ and Cu²⁺ dramatically shortened the clotting time in the presence of NTA-lipids in a concentration-dependent manner (FIG. 12C). Ni²⁺ performed substantially better in this assay than did Cu²⁺. Ni²⁺ exhibited the greatest activity (shortest clot time) when incorporated into the modified aPTT reagent at 10 to 25 μM.

Ni²⁺ bound to other immobilized supports exhibits procoagulant activity by activating the contact pathway of blood clotting. This was tested by using Ni Sepharose 6 Fast Flow beads (Amersham Biosciences), which contains Ni²⁺ ions chelated to an NTA moiety that is covalently attached to cross-linked agarose beads (Ni-NTA beads). The procoagulant activity of Ni-NTA beads was tested using a 96-well plate reader because the agarose beads interfered with the ball bearing detection system in the ST4 coagulometer. As a comparator, the ability of 15% Ni-lipids was tested in this same test system. The samples containing 15% Ni-lipids clotted almost immediately (too quickly to be measured by the microplate reader), indicating their superiority as contact pathway activators (data not shown). The clotting time of plasma in the absence of activator was 311 sec (Table 3). The Ni-NTA beads shortened the clotting time of the plasma to 126 sec., which, while a significant shortening, nevertheless is substantially longer than was observed for 15% Ni-lipids. This indicates that Ni-NTA beads have measurable procoagulant activity, but it also indicates that they are inferior to Ni-lipids. As a control to make certain that the agarose beads themselves were not procoagulant, an aliquot of beads was stripped of bound nickel ions by exposure to EDTA (the EDTA was subsequently removed by extensive washing). These stripped NTA beads exhibited negligible procoagulant activity.

TABLE 3
Clot time for pooled normal plasma in the presence
of various forms of Ni-NTA to activate clotting

| Contact Pathway Activator | Clot Time (sec.) |
|---|---|
| No Activator | 311 |
| Ni-NTA beads | 126 |
| Stripped NTA beads | 236 |

Ni-NTA beads can be used to deplete plasma of contact factors

Because the Ni-lipids activate the contact pathway, at least one of the factors in the contact pathway must be a Ni²⁺-binding protein. Initial studies were carried out to determine if normal pooled plasma could be depleted of factors in the contact pathway through adsorption to Ni-NTA beads. The plasma was incubated with Ni-NTA beads for 30 minutes at ambient temperature and then the beads were removed from the plasma by filtration (depleted plasma). We measured the clotting times of depleted plasma using both a modified PT assay (with rTF/PCPS as the thromboplastin reagent) and an aPTT assay (with the Diagnostica Stago aPTT reagent). Clotting times with depleted plasma were compared to those with normal pooled plasma (not treated with Ni-NTA beads) (Table 4). In the PT assay, the clotting time of depleted plasma was shorter than that of normal plasma. In the aPTT assay, the clotting time of depleted plasma was substantially longer than that of normal pooled plasma. This result indicates that critical contact factor(s) can be depleted from plasma by adsorption onto Ni-beads.

TABLE 4
Clot times comparing depleted plasma with pooled
normal plasma using both the PT and aPTT assays

| | PT (sec.) | aPTT (sec.) |
|---|---|---|
| Pooled normal plasma | 16.2 ± 0.2 | 35.2 ± 1.5 |
| Depleted plasma | 16.2 ± 0.2 | 83.7 ± 17.6 |

Additional Oligohistidine-Tagged sTF Variants

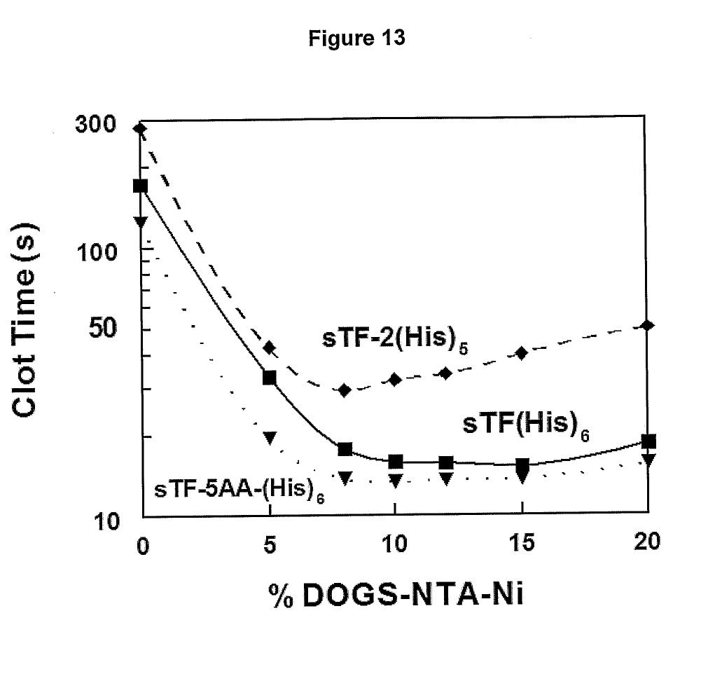

Studies were also performed using two new oligohistidine-tagged sTF variants: sTF-2(His)₅ and sTF-5AA-(His)₆ (see FIG. 1 for amino acid sequences). PT assays were performed with all three versions of oligohistidine-tagged sTF (at 0.15 μg/ml) using 50 μM SUVs composed of varying amounts of DOGS-NTA-Ni (with 5% PS, 30% PE, and the balance being made up of PC) (FIG. 13). These studies show that the sTF-2(His)₅ construct has reduced procoagulant activity relative to sTF(His)₆, while the sTF-5AA-(His)₆ construct has increased procoagulant activity relative to sTF(His)₆.

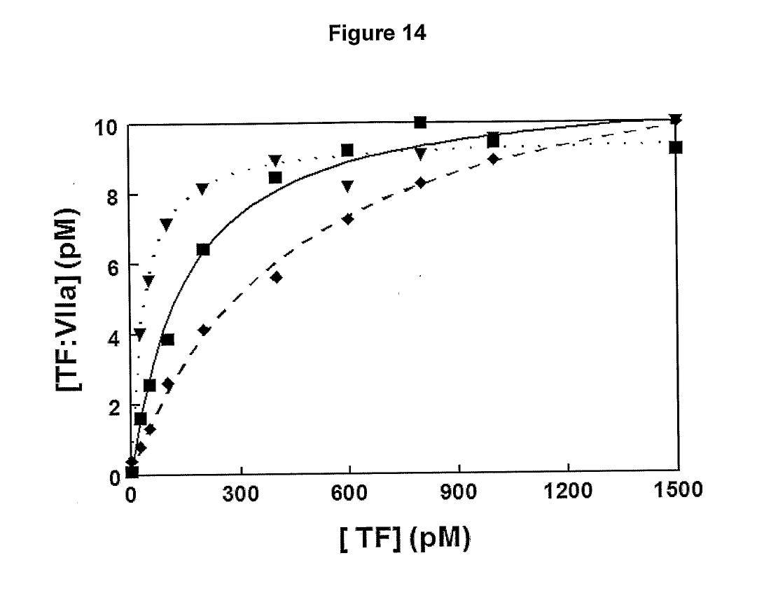

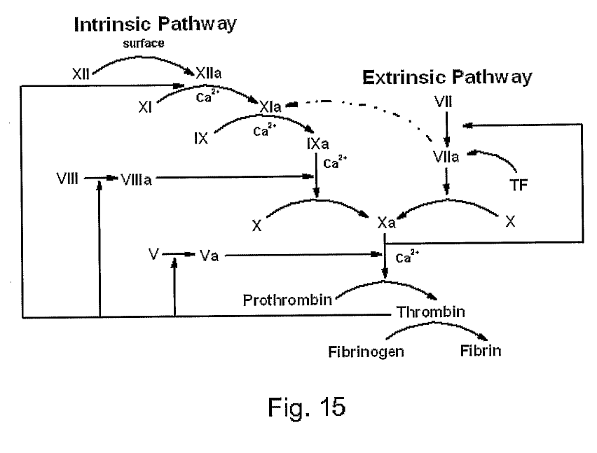

The binding affinity of each variant for VIIa was also measured, using the same conditions earlier used to measure K_{d} values for sTF(His)₆ (see FIG. 14 for binding isotherms, and Table 5 for K_{d}, values). These studies demonstrated that the sTF-2(His)₅ construct binds more weakly to factor VIIa than does the sTF(His)₆ construct. In contrast, the sTF-5AA-(His)₆ construct binds considerably more tightly to factor VIIa than does the sTF(His)₆ construct. These results indicate that the sTF-5AA-(His)₆ construct is superior to the sTF(His)₆ construct.

TABLE 5
Dissociation constants for VIIa binding to the his-tagged sTF variants

| TF | K_{d} (pM) |
|---|---|
| sTF(His)₆ | 169 |
| sTF-2(His)₅ | 440 |
| sTF-5AA-(His)₆ | 31 |

When VIIa binds to TF, its rate of X activation increases dramatically, so this can be used as a convenient readout for TF:VIIa complex formation. Using this approach, we found that VIIa bound with extremely high affinity to the combination of sTF-5AA-(His)₆ plus NiPCPS (phospholipid vesicles containing 15% DOGS-NTA-Ni, 65% PC, 20% PS), with a K_{d} of 10.8 pM (Table 6). This was essentially identical to its affinity for recombinant membrane-bound tissue factor (membTF) in PCPS liposomes (K_{d}=10.0 pM). Therefore, when the isolated TF ectodomain was attached to the membrane surface via interaction with metal-chelating lipids, its VIIa binding ability was indistinguishable from membTF that spanned the lipid bilayer.

A stringent test of TF:VIIa function is its ability to support the activation of its natural protein substrate, X. This requires that TF be incorporated into a suitable phospholipid membrane (i.e., one containing negatively charged phospholipids). The isolated TF ectodomain, on the other hand, supports orders of magnitude lower rates of X activation than does membTF—even in the presence of PCPS liposomes—because sTF is not anchored in the membrane [4,9]. The ability of various forms of TF to support the activation of X was compared under equivalent concentrations of enzyme (500 pM VIIa), cofactor (4 pM TF) and liposomes (50 μM total lipid). The combination of sTF-5AA-(His)₆ and NiPCPS supported rates of X activation by VIIa that were comparable to those obtained using membTF in PCPS liposomes (Table 6). The k_{cat} values for the two forms of the TF:VIIa complex were similar, while the Kₘ of VIIa bound to sTF-5AA-(His)₆ plus NiPCPS for X was actually lower than with membTF in PCPS, leading to a slightly higher overall catalytic efficiency (k_{cat}/Kₘ). The high enzymatic activity of Vila bound to sTF-5AA-(His)₆ was dependent on both nickel-chelating lipids and the oligohistidine tag on sTF, since mixing either sTF-5AA-(His)₆ with PCPS liposomes, or sTF with NiPCPS liposomes, supported negligible rates of X activation by VIIa.

An additional function of TF is to promote the autoactivation of VII in a reaction that is dependent on TF surface density [25,50]. In contrast, sTF fails to support this reaction [11]. Under conditions of identical TF surface densities, both membTF in PCPS and sTF-5AA-(His)₆ plus NiPCPS supported VII autoactivation with comparable rate constants (Table 6). Together, these findings show that it is not necessary for the TF ectodomain to be covalently attached to a membrane anchor to achieve wild-type levels of TF activity; reversible attachment of sTF to the membrane surface via metal-chelating lipids is functionally equivalent to conventional membrane anchoring.

TABLE 6
Binding and kinetic constants for TF:VIIa complexes

| | | VIIa | | | | VII |
|---|---|---|---|---|---|---|
| | | binding | X activation | | | autoactivation |
| | | K_{d} | Kₘ | k_{cat} | k_{cat}/Kₘ | k_{2D} |
| TF | Lipid | (pM) | (nM) | (s⁻¹) | (μM⁻¹ s⁻¹) | (m² mol⁻¹ s⁻¹) |
| membTF^{a} | PCPS | 10.0 ± 4.4 | 59 ± 0.88 | 3.5 ± 0.38 | 60.2 ± 6.35 | 3.4 (±0.15) × 10⁷ |
| | liposomes | | | | | |
| sTF- | NiPCPS | 10.8 ± 4.2 | 38 ± 3.8 | 3.3 ± 0.43 | 87.1 ± 12.3 | 2.9 (±0.37) × 10⁷ |
| 5AA- | liposomes | | | | | |
| (His)₆^{a} | | | | | | |
| sTF- | Immobilized | n.d^{c} | 66 ± 6.4 | 4.1 ± 1.4 | 63.1 ± 22.3 | n.d^{c} |
| 5AA- | 10% DOGS- | | | | | |
| (His)₆^{b} | NTA-Ni, | | | | | |
| | 20% PS, | | | | | |
| | 70% PC. | | | | | |

^{a}Purified proteins.
^{b}Captured from crude culture supernatants.
^{c}Not determined.

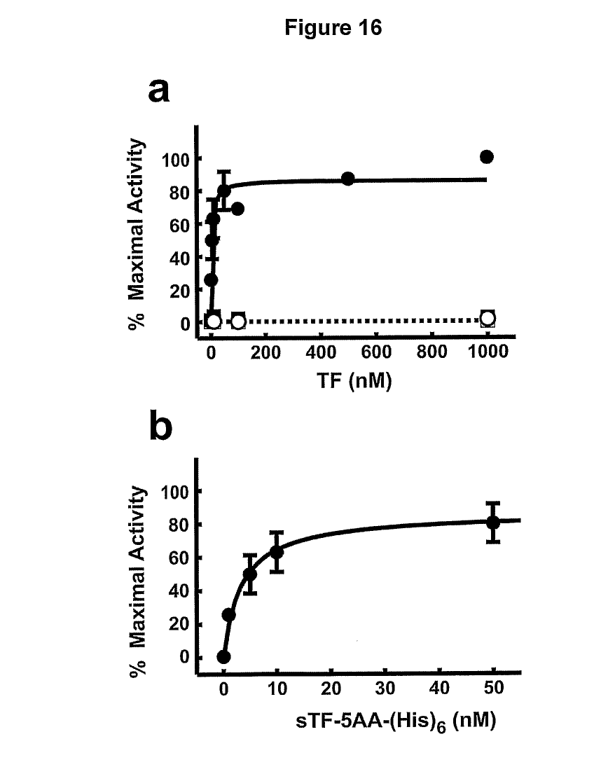

The experiments described above used purified sTF-5AA-(His)₆. We reasoned that immobilized lipid bilayers containing DOGS-NTA-Ni should be able to capture sTF-5AA-(His)₆ from crude mixtures, simultaneously isolating and membrane-anchoring this protein in one quick step. Although expression of sTF-5AA-(His)₆ in our E. coli expression system is targeted to the periplasmic space, significant quantities accumulate in the medium of overnight cultures: Typically, 55 μg/ml (2.0 μM) sTF-5AA-(His)₆ as measured by ELISA. Crude culture supernatants were therefore diluted tenfold with buffer and incubated in the wells of a polystyrene 96-well plate that had previously been coated with a lipid mixture containing DOGS-NTA-Ni, PS and PC. After washing away unbound proteins, the wells were treated with VIIa and the rate of X activation was measured. Because VIIa is a very poor activator of X in the absence of TF and a suitable phospholipid membrane, this assay is a stringent test both of the ability of the immobilized lipids to capture sTF-5AA-(His)₆ and of the functional state of the resulting TF:VIIa:membrane complexes. It was found that when sTF-5AA-(His)₆ was captured in this way from culture supernatants it robustly supported X activation by VIIa, with apparent Kₘ and k_{cat} values that were comparable to those of membTF in PCPS liposomes (Table 6). Under these assay conditions, the concentration of purified sTF-5AA-(His)₆ required to support half-maximal rates of X activation (EC₅₀) was 6.2±4.1 nM (FIG. 16), a concentration that is more than two orders of magnitude below the sTF-5AA-(His)₆ concentration in our E. coli culture supernatants. Both the oligohistidine tag on sTF and the presence of nickel-chelating lipids were required, since neither combinations of sTF-5AA-(His)₆ with immobilized PCPS nor of sTF with immobilized nickel-chelating lipid mixtures yielded detectable levels of X activation, even at sTF concentrations as high as 1 μM (FIG. 16).

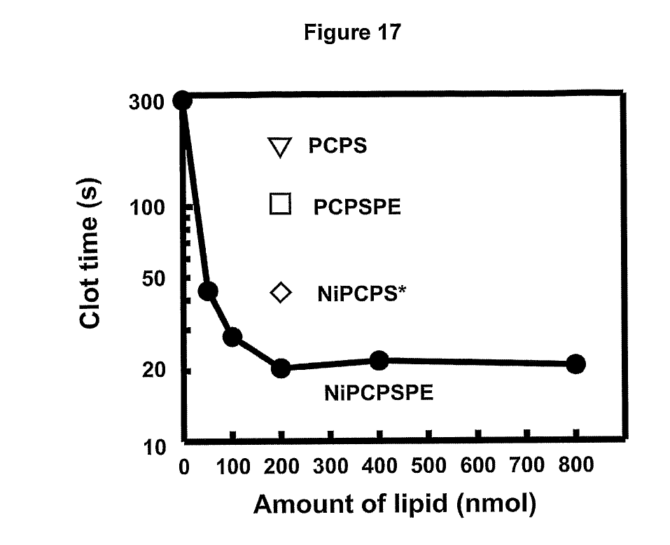

Mixtures of DOGS-NTA-Ni and phospholipids were dried-down in the wells of a polystyrene coagulometer cuvette, the cuvettes were washed to remove any non-immobilized lipid, and then sTF-5AA-(His)₆ was added to the wells. Clotting tests were subsequently conducted by adding calcium ions and plasma to wells. It was found that clot times below 25 s could be achieve using 20 nM sTF-5AA-(His)₆ in cuvettes that had received 200 to 800 nmol NiPCPSPE (phospholipid vesicles comprising 10% DOGS-NTA-Ni, 47.5% PC, 12.5% PS, 30% PE) per well (FIG. 17). Control experiments were conducted using other lipid compositions, which were all dried down onto the cuvette well at a single amount of lipid per well (200 nmol). Markedly longer clotting times were observed when the lipids lacked DOGS-NTA-Ni (PCPS or PCPSPE; FIG. 17). Lipid mixtures containing DOGS-NTA-Ni but lacking PE had moderately prolonged clotting times (NiPCPS*; FIG. 17). This experiment demonstrates that the combination of immobilized NiPCPSPE and sTF-5AA-(His)₆ function as a potent thromboplastin reagent. They also demonstrate that DOGS-NTA-Ni is required for efficient functioning of sTF-5AA-(His)₆ as a procoagulant agent, and that PE enhances but is not absolutely required for the procoagulant activity of sTF-5AA-(His)₆.

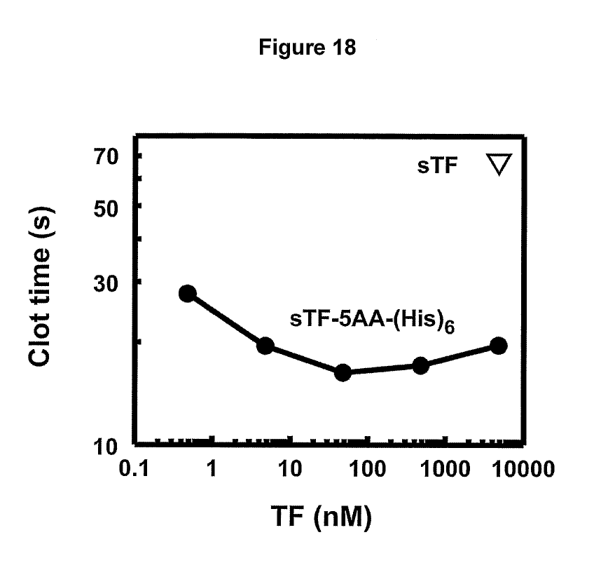

The dependence of clotting time on sTF-5AA-(His)₆ concentration was investigated. Using 200 nmol dried NiPCPSPE per well, we found that clotting times below 20 s could be obtained with sTF-5AA-(His)₆ concentrations ranging from 4.8 to 4800 nM (FIG. 18). The shortest clotting time in this experiment (16.3 s) was obtained using 48 nM sTF-5AA-(His)₆. In contrast, sTF without the oligohistidine tag exhibited much longer clotting times, even when used at very high concentrations (4800 nM; FIG. 18). This demonstrates that the oligohistidine tag of sTF-5AA-(His)₆ is required for it to function as an efficient thromboplastin reagent in the presence of immobilized NiPCPSPE.

Materials & Methods

Materials—Pooled normal human plasma and individual factor-deficient plasmas (deficient in factors V, VII, VIII, IX, X, XI, XII, or prekallikrein) were purchased from George King Bio-Medical. Kininogen (HK)-deficient plasma was purchased from Affinity Biologicals. Chicken egg phosphatidylcholine (PC), porcine brain phosphatidylserine (PS), bovine liver phosphatidylethanolamine (PE) and DOGS-NTA-Ni were purchased from Avanti Polar Lipids, Inc. The lipids were supplied dissolved in chloroform and were stored under nitrogen at −20° C. until needed. Chromozym® t-PA (N-methylsulfonyl-D-Phe-Gly-Arg-4-nitranilide acetate) was purchased from Roche Applied Science. S-2222 was purchased from DiaPharma. Bio-Beads® SM-2 adsorbent were purchased from BioRad Laboratories. Octaethylene glycol monododecyl ether (C₁₂E₈) was purchased from Fluka. Recombinant human VIIa was purchased from American Diagnostica and plasma-derived factor X was from Enzyme Research Laboratories. Commercial PT reagent (STA-Neoplastine CI Plus) and aPTT reagent (STA-PTT-Automate 5) were purchased from Diagnostica Stago. Ni Sepharose 6 Fast Flow beads were purchased from Amersham Biosciences. Recombinant human rTF and sTF were expressed in E. coli cells and purified as previously described [6, 21]. Bovine serum albumin (BSA) was from Calbiochem (La Jolla, Calif.). ST4 coagulometer cuvettes and the STart 4 coagulometer were from Diagnostica Stago (Parsippany, N.J.). Spectrozyme Xa substrate (methoxycarbonyl-D-cyclohexylglycyl-Gly-Arg-4-nitroanilide acetate) and recombinant human VIIa were from American Diagnostica, Inc. (Stamford, Conn.). Purified plasma-derived VII, X and factor Xa (Xa) were from Enzyme Research Laboratories (South Bend, Ind.). Antifoam C was from Sigma (Sigma-Aldrich, St. Louis, Mo.).

Production of Oligohistidine-tagged sTF—Three different versions of oligohistidine-tagged sTF were generated by mutating the vector containing the sequence for sTF. The expression vector for producing the variants in E. coli is a modified version of plasmid pET26b(+) (Novagen). All three versions encoded the following amino acid sequences (listed from N-terminal to C-terminal):

1. A bacterial leader peptide (pelB) for targeting of the recombinant protein to the periplasmic space of E. coli. The pelB leader peptide is removed by E. coli cells during synthesis of the protein.

2. A short peptide epitope (AEDQVDPRLIDGKS) on the N-terminus of mature, recombinant sTF for affinity purification using immobilized HPC4 antibody. Numerous experiments have shown that the presence of this short peptide on the N-terminus of sTF has no effect on its function [6].

3. The extracellular domain of human TF, consisting of amino acids 1-217 or 1-219 of the mature human TF sequence (numbered according to Morrissey et al. [22]).

The sequence at the C-terminal end of sTF differs in the three variants (FIG. 1). sTF(His)₆ replaces the final two amino acids of sTF with six histidine residues. sTF-2(His)₅ is similar to sTF(His)₆ except that it contains five histidine residues, an eight amino acid-long spacer, and five more histidine residues. sTF-5AA-(His)₆ retains the last two amino acids of sTF, then contains a 5 amino acid-long spacer, followed by six histidine residues. All three variants were expressed in E. coli BL21(DE3) cells and purified using an HPC4 immunoaffinity column as previously described for sTF [6], with the following minor modifications.

Bacterial pellets were obtained via centrifugation as previously described in Rezaie, et al. The pellet was washed with cell wash buffer (10 mM Tris-HCl, pH 7.5, 30 mM NaCl, 0.5 mM EDTA, pH 8.0), centrifuged as previously described, and washed and centrifuged a second time. The washed pellet was resuspended in spheroplast buffer (100 mM Tris-HCl, pH 8.0, 0.5 mM EDTA, pH 8.0, 1 mM MgCl₂, 500 mM Sucrose) plus 0.2 mM PMSF. A pellet was collected via centrifugation and incubated at ambient temperature for 10 min. The pellet was resupended in cold H₂O and incubated on ice for 5 minutes. MgCl₂ was added to the suspension at a final concentration of 1 mM, and the suspension was centrifuged again. The supernatant was collected and chromatographed as described previously [6].

Vesicle preparation—Small unilamellar phospholipid vesicles (SUV) were prepared by three different methods. In all three methods, a total of 2.6 mmol of the desired lipid mixture was dried down under a stream of dry nitrogen, followed by 1 hr of additional drying under high vacuum to remove any traces of chloroform. Unless otherwise noted, phospholipid vesicles were prepared by adapting the Bio-Bead method [17]. In this case, the dried-down lipid mixture was resuspended in 1 ml HBS (20 mM HEPES-NaOH buffer pH 7.4, 100 mM NaCl, 0.1% NaN₃) plus 6 mM C₁₂E₈, for 40 minutes at room temperature. The C₁₂E₈ was then removed by incubating the solution for 1.5 hr at room temperature with 400 mg of Bio-Beads [17]. The other two methods for vesicle preparation were sonication and extrusion. For either of these two methods, the dried-down lipid mixtures were resuspended in 1 ml HBS, giving a final lipid concentration of 2.6 mM. The turbid lipid suspensions were then either sonicated in a bath sonicator until they became visually clear, thereby generating SUVs, or were extruded repeatedly through 100 nm polycarbonate filters using the Avestin LiposoFast vesicle extruder. In all cases, the lipid mixtures consisted of varying amounts of PS, DOGS-NTA-Ni, PE and sufficient PC to make the total lipid content equal to 2.6 mmol. SUVs consisting of 20 mol % PS and 80 mol % PC are referred to as PCPS. Unless otherwise noted, SUVs containing 5% PS, 40% PE, and varying amounts of DOGS-NTA-Ni and PC are referred to as Ni-lipids and are indicated by their DOGS-NTA-Ni content. Thus, 15% Ni-lipids refers to SUVs containing 5% PS, 40% PE, 15% DOGS-NTA-Ni, and 40% PC.

Thromboplastin reagents—To prepare a conventional thromboplastin reagent, rTF was relipidated into phospholipid vesicles composed of 20 mol % PS, 80 mol % PC (rTF/PCPS) at a 8700:1 molar ratio of phospholipid to rTF as described [17,23]. rTF/PCPS preparations were then diluted to the desired final rTF concentration in TBSA (50 mM Tris-HCl buffer, pH 7.5, 100 mM NaCl, 0.1% bovine serum albumin, 0.1% NaN₃).

To prepare a modified thromboplastin reagent, SUVs and either sTF or one of the oligohistidine-tagged sTF variants were diluted to the desired concentrations in TA (50 mM Tris-HCl buffer, pH 7.5, 0.1% bovine serum albumin, 0.1% NaN₃).

PT clotting assay—PT assays were performed in a model ST4 coagulometer (Diagnostica Stago) by pipetting 50 μl of 25 mM CaCl₂ and 50 μl diluted thromboplastin reagent into a coagulometer cuvette and allowing the mixture to warm to 37° C. for 2 min. Clotting was then initiated by pipetting 50 μl pre-warmed, pooled normal plasma into the cuvette and the time to clot formation recorded.

aPTT clotting assay—aPTT clotting assays were also carried out in a model ST4 coagulometer (Diagnostica Stago). The aPTT assays were performed by pipetting 50 μl of an aPTT reagent and 50 μl pooled normal plasma into a coagulometer cuvette and incubating the mixture for 3 min at 37° C. 50 μl prewarmed 25 mM CaCl₂ was then pipetted into the cuvette and the time to clot formation was recorded.

Measurement of Allosteric Activation of VIIa—The ability of TF to allosterically activate factor VIIa was carried out measuring the enzymatic activity of factor VIIa by a chromogenic assay. Reaction mixtures containing VIIa and various concentrations of TF (or sTF) were prepared in HBSAC (HBS plus 0.1% bovine serum albumin and 5 mM CalCl₂). The reaction was initiated by the addition of the chromogenic substrate, Chromozym® t-PA (ChtPA) in a flat bottom, 96-well plate. Typical reaction conditions for all forms of TF were 15 nM VIIa, 0-50 nM TF (rTF is relipidated in PCPS vesicles; sTF variants were mixed with phospholipid vesicles at a final concentration of 50 μM phospholipid), and 1 mM ChtPA in HBSAC at a final volume of 100 μl. Change in A₄₀₅ was monitored at ambient temperature in a VERSAmax microplate reader (Molecular Devices), reading every 30 seconds for 20 minutes.

Measurement of the K_{d} for VIIa binding to TF—The binding affinity of VIIa for various forms of TF was measured using the TF-dependent increase in the rate of X activation as the readout for TF:VIIa complex formation. Activation of X by the TF:VIIa complex was measured by adapting the continuous chromogenic assay of Fiore et al. [9]. Reaction mixtures containing Vila and either rTF/PCPS or sTF+SUVs were prepared in HBSAC. Reactions were initiated by adding a mixture of X and the chromogenic substrate, S-2222, in a flat bottom, 96-well plate. Change in A₄₀₅ was monitored at ambient temperature in a VERSAmax microplate reader (Molecular Devices), sampling every 15 sec for 20 min. Initial rates of X activation were determined by fitting a second-order polynomial to the A₄₀₅ data as described [9,24]. The quadratic ligand binding equation was fit to the rate data as described [24] to derive apparent K_{d} values for the binding of VIIa to TF. For experiments using relipidated rTF, typical reaction mixtures containing 0.25 μM VIIa and 20 nM X in HBSAC were incubated with increasing concentrations of rTF/PCPS. For experiments using sTF, reaction mixtures were modified to contain 400 pM VIIa, 100 μM PCPS vesicles, 20 nM X, and varying sTF concentrations. For experiments using the oligohistidine-tagged forms of sTF, reaction mixtures were modified to contain 10 pM VIIa, Ni-lipids (50 μM total lipid), 20 nM X, and varying concentrations of oligohistidine-tagged sTF variants.

Measurements of clot formation in the plate reader—In this experiment pooled normal plasma supplemented with 50 μM PCPS vesicles was mixed with various amounts of Ni-NTA beads (Ni Sepharose 6 Fast Flow) in TA, and incubated for 3 minutes at 37° C. 80 μl aliquots of this mixture were added to 20 μl of pre-warmed (37° C.) 53 mM CaCl₂ in a flat bottom, 96-well plate. Change in A₄₀₅ was monitored at 37° C. in a VERSAmax microplate reader over 20 minutes with measurements taken every 30 sec. Time to half maximal absorbance was used as the clot time.

Factor X activation using liposomes. Initial rates of X activation were measured at ambient temperature in multiwell plates using a discontinuous chromogenic assay [4] modified as follows: Reaction mixtures in HBSAC contained 500 pM VIIa, varying X, and either 4 pM membTF in PCPS (plus 50 μM PCPS) or 4 pM sTF-5AA-(His)₆ plus 50 μM NiPCPS. At varying times, 20 μl aliquots were removed into a 96-well plate containing 100 μl Stop Buffer 1 (40 mM Mes-NaOH pH 5.8, 12 mM EDTA, 50 mM NaCl, 0.25% Triton X-100, 0.1% NaN3, 0.012% Antifoam C) at 4° C. After warming the stopped reactions to room temperature, Xa was detected by adding 60 μl of 1.5 mM Spectrozyme Xa in HBSAC plus 0.6 M Tricine-NaOH, pH 8.4 and quantifying change in A405. Amounts of Xa generated were determined by comparison to a standard curve with purified Xa.

Factor X activation using immobilized lipids. All steps were conducted at ambient temperature. Lipid mixtures were dried down in a borosilicate glass test tube under a gentle stream of dry nitrogen gas to remove the solvent (chloroform), then redissolved in n-hexane at a total lipid concentration of 2 mM. Each well of a 96-well polystyrene plate (Costar 9018 high-binding plates, Corning, Inc., Corning, N.Y.) received 60 nmol total lipid, and the hexane was allowed to evaporate in a fume cupboard. The wells were then incubated for 1 hr with 100 μl TBSA (50 mM Tris-HCl pH 7.5, 100 mM NaCl, 0.02% NaN3, 1% bovine serum albumin), aspirated and washed thrice with TBS (TBSA without albumin). Wells were incubated for 1 hr with 100 μl of the indicated concentration of sTF or sTF-5AA-(His)₆ in HBSA (HBSAC without calcium), then aspirated and washed thrice with TBS. Wells were incubated for 1 hr with 100 μl of 5 pM factor VIIa in HBSAC, after which reactions were initiated by adding 100 μl of 1 mM Spectrozyme Xa substrate and the indicated X concentrations HBSAC. Change in A405 was quantified and the amount of Xa generated determined by comparison to a standard curve.

Factor VII autoactivation. Rates of VII autoactivation were measured essentially as described [25]. Equimolar concentrations of VII and TF (15 nM each) were incubated in HBSAC at 37° C. At various time points, 20 μl aliquots were removed to a 96-well plate containing 60 μl Stop Buffer II (0.1 M Tricine-NaOH, pH 8.4, 6.7 mM CaCl₂, 0.1% bovine serum albumin, 0.1% Triton X-100, 0.05% NaN₃ and 100 nM sTF). A 20 μl aliquot of 5 mM Chromozym t-PA substrate was added and the change in A405 was monitored at ambient temperature. Two-dimensional second-order rate constants (k2D) were determined as described [25].

TF clotting assay. Thromboplastin reagents for clotting assays were prepared using membTF in PCPS, NiPCPS, or NiPCPSPE liposomes in Low Salt TBSA (TBSA containing 10 mM NaCl instead of 100 mM) to which additional appropriate liposomes were added to achieve a total lipid concentration of 100 μM. Thromboplastin reagents containing either sTF or sTF-5AA-(His)₆ were likewise prepared in Low Salt TBSA plus 100 μM PCPS, NiPCPS, or NiPCPSPE liposomes. Clotting assays were performed in a STart 4 coagulometer (Diagnostica Stago, Parsippany, N.J.). Briefly, 50 μl aliquots each of 25 mM CaCl₂ and thromboplastin reagent were incubated together for 120 s in a coagulometer cuvette at 37° C. A 50 μl aliquot of pre-warmed pooled normal human plasma was then added, and the time to clot formation was measured. By modifying the clotting assay to add plasma last, activation of the contact pathway was minimized and the clotting times were dependent upon TF activity. A unit of TF activity was defined as the amount of TF in the final 150 μl clotting reaction that yields a 50 s clot time.

Clotting assay with immobilized lipids—Lipid mixtures in chloroform were first dried down in a borosilicate glass test tube under a gentle stream of dry nitrogen gas to remove the solvent, after which the dried lipids were redissolved in n-hexane at a total lipid concentration of 0.3 to 5.3 mM. Complete lipid mixtures were termed NiPCPSPE and consisted of 10% DOGS-NTA-Ni, 12.5% PS, 30% PE, and 47.5% PC. Lipid mixtures lacking one of each of these components were also prepared for control experiments, whose compositions are as follows: PCPSPE contained 12.5% PS, 30% PE, and 57.5% PC; NiPCPS* contained 10% DOGS-NTA-Ni, 12.5% PS, and 77.5% PC; and PCPS contained 12.5% PS and 87.5% PC.

Each well of an ST4 cuvette received 150 μl of lipid solution in hexane (containing 50 to 800 nmol total lipid per well). The hexane was then allowed to completely evaporate in a fume cupboard at room temperature. Next, the wells were washed thrice with TBS (50 mM Tris-HCl pH 7.5, 100 mM NaCl, and 0.02% NaN₃). Each well then received 50 μl of a solution of the indicated concentration of sTF or sTF-5AA-(His)₆ in TA (50 mM Tris-HCl pH 7.5, 0.1% BSA, 0.1% NaN₃), after which the wells were covered with parafilm and incubated at room temperature for 1 hr. The parafilm was then removed from the cuvettes and they were transferred to a STart 4 coagulometer which had been preheated to 37° C. A 50 μl aliquot of 25 mM CaCl₂ was added per well and the cuvettes were incubated for 2 min at 37° C., after which a 50 μl aliquot of pre-warmed (37° C.) pooled normal human plasma was added and the time to clot formation was measured.

### REFERENCES

1 Morrissey J H. Tissue factor and factor VII initiation of coagulation. In: Colman R W, Hirsh J, Marder V J, Clowes A W, George J N, editors. Hemostasis and Thrombosis: Basic Principles and Clinical Practice. Philadelphia: Lippincott Williams & Wilkins, 2001: 89-101.
2 Camerer E, Prydz H. Notes on the cell biology of tissue factor. Haemostasis 1996; 26:25-30.
3 Paborsky L R, Caras I W, Fisher K L, Gorman C M. Lipid association, but not the transmembrane domain, is required for tissue factor activity. Substitution of the transmembrane domain with a phosphatidylinositol anchor. J Biol Chem 1991; 266:21911-21916.
4 Neuenschwander P F, Morrissey J H. Roles of the membrane-interactive regions of factor VIIa and tissue factor. The factor VIIa Gla domain is dispensable for binding to tissue factor but important for activation of factor X. J Biol Chem 1994; 269:8007-8013.
5 Waxman E, Ross J B A, Laue T M, Guha A, Thiruvikraman S V, Lin T C, Konigsberg W H, Nemerson Y. Tissue factor and its extracellular soluble domain: The relationship between intermolecular association with factor VIIa and enzymatic activity of the complex. Biochemistry 1992; 31:3998-4003.
6 Rezaie A R, Esmon C T, Morrissey J H. Expression and Purification of Recombinant Soluble Tissue Factor. U.S. Pat. NO. 5,298,599. The entire contents of this patent are hereby incorporated by reference.
7 Shigematsu Y, Miyata T, Higashi S, Miki T, Sadler J E, Iwanaga S. Expression of human soluble tissue factor in yeast and enzymatic properties of its complex with factor VIIa. J Biol Chem 1992; 267:21329-21337.
8 Waxman E, Laws W R, Laue T M, Nemerson Y, Ross J B A. Human factor VIIa and its complex with soluble tissue factor: Evaluation of asymmetry and conformational dynamics by ultracentrifugation and fluorescence anisotropy decay methods. Biochemistry 1993; 32:3005-3012.
9 Fiore M M, Neuenschwander P F, Morrissey J H. The biochemical basis for the apparent defect of soluble mutant tissue factor in enhancing the proteolytic activities of factor VIIa. J Biol Chem 1994; 269:143-149.
10 Ruf W, Rehemtulla A, Morrissey J H, Edgington T S. Phospholipid-independent and -dependent interactions required for tissue factor receptor and cofactor function. J Biol Chem 1991; 266:2158-2166.
11 Neuenschwander P F, Morrissey J H. Deletion of the membrane anchoring region of tissue factor abolishes autoactivation of factor VII but not cofactor function. Analysis of a mutant with a selective deficiency in activity. J Biol Chem 1992; 267:14477-14482.
12 Nemerson Y, Repke D. Tissue factor accelerates the activation of coagulation factor VII: The role of a bifunctional coagulation cofactor. Thromb Res 1985; 40:351-358.
13 Morrissey J H, Macik B G, Neuenschwander P F, Comp P C. Quantitation of activated factor VII levels in plasma using a tissue factor mutant selectively deficient in promoting factor VII activation. Blood 1993; 81:734-744.
14 Hirsh J, Fuster V, Ansell J, Halperin J L. American Heart Association/American College of Cardiology Foundation guide to warfarin therapy. Circulation 2003; 107:1692-1711.
15 Bader R, Mannucci P M M, Tripodi A, Hirsh J, Keller F, Solleder E M, Hawkins P, Peng M, Pelzer H, Teijidor L M, Ramirez I F, Kolde H-J. Multicentric evaluation of a new PT reagent based on recombinant human tissue factor and synthetic phospholipids. Thromb Haemost 1994; 71:292-299.
16 Tripodi A, Arbini A, Chantarangkul V, Mannucci P M. Recombinant tissue factor as substitute for conventional thromboplastin in the prothrombin time test. Thromb Haemost 1992; 67:42-45.
17 Smith S A, Morrissey J H. Rapid and efficient incorporation of tissue factor into liposomes. J Thromb Haemost 2004; 2:1155-1162.
18 Banner D W, D'Arcy A, Chène C, Winkler F K, Guha A, Konigsberg W H, Nemerson Y, Kirchhofer D. The crystal structure of the complex of blood coagulation factor VIIa with soluble tissue factor. Nature 1996; 380:41-46.
19 Terpe K. Overview of tag protein fusions: from molecular and biochemical fundamentals to commercial systems. Appl Microbiol Biotechnol 2003; 60:523-533.
20 Darst S A. A new twist on protein crystallization. Proc Natl Acad Sci USA 1998; 95:7848-7849.
21 Neuenschwander P F, Bianco-Fisher E, Rezaie A R, Morrissey J H. Phosphatidylethanolamine augments factor VIIa-tissue factor activity: Enhancement of sensitivity to phosphatidylserine. Biochemistry 1995; 34:13988-13993.
22 Morrissey J H, Fakhrai H, Edgington T S. Molecular cloning of the cDNA for tissue factor, the cellular receptor for the initiation of the coagulation protease cascade. Cell 1987; 50:129-135.
23 Smith S A, Morrissey J H. Properties of recombinant human thromboplastin that determine the International Sensitivity Index (ISI). J Thromb Haemost 2004; 2:1610-1616.
24 Fiore M M, Neuenschwander P F, Morrissey J H. An unusual antibody that blocks tissue factor/factor VIIa function by inhibiting cleavage only of macromolecular substrates. Blood 1992; 80:3127-3134.
25 Neuenschwander P F, Fiore M M, Morrissey J H. Factor VII autoactivation proceeds via interaction of distinct protease-cofactor and zymogen-cofactor complexes. Implications of a two-dimensional enzyme kinetic mechanism. J Biol Chem 1993; 268:21489-21492.
26 Lauer S A, Nolan J P. Development and characterization of Ni-NTA-bearing microspheres. Cytometry 2002; 48:136-145.
27 Cornell B A, Krishna G, Osman P D, Pace R D, Wieczorek L. Tethered-bilayer lipid membranes as a support for membrane-active peptides. Biochem Soc Trans 2001; 29:613-617.
28 Gemmell C H, Turitto V T, Nemerson Y. Flow as a regulator of the activation of factor X by tissue factor. Blood 1988; 72:1404-1406.
29 Gemmell C H, Nemerson Y, Turitto V. The effects of shear rate on the enzymatic activity of the tissue factor-factor VIIa complex. Microvasc Res 1990; 40:327-340.
30 Morrissey J H. Tissue factor: an enzyme cofactor and a true receptor. Thromb Haemost 2001; 86:66-74.
31 Massignon D, Moulsma M, Bondon P, Debize G, Abidi H, Buttin T, Bon C, Pillonchery G, Coeur P. Prothrombin time sensitivity and specificity to mild clotting factor deficiencies of the extrinsic pathway: evaluation of eight commercial thromboplastins. Thromb Haemost 1996; 75:590-594.
32 Kitchen S, Jennings I, Woods T A, Walker I D, Preston F E. Two recombinant tissue factor reagents compared to conventional thromboplastins for determination of international normalised ratio: a thirty-three-laboratory collaborative study. The Steering Committee of the UK National External Quality Assessment Scheme for Blood Coagulation. Thromb Haemost 1996; 76:372-376.
33 Kemball-Cook G, Garner I, Imanaka Y, Nishimura T, O'Brien D P, Tuddenham E G, McVey J H. High-level production of human blood coagulation factors VII and XI using a new mammalian expression vector. Gene. 1994 Feb. 25; 139(2):275-9.
34 Van Den Besselaar, A. M., Tripodi, A., Poller, L. WHO guidelines for thromboplastins and plasma used to control oral anticoagulation therapy. Annex 3. World Health Organ Tech Rep Ser. 1999; 889: 64-93.
35 Jeong, S. W., O'Brien, D. F. J Org Chem. 2001 Jul. 13; 66(14): 4799-802.
36 M. S. Kent, H. Yim, D. Y. Sasaki, J. Majewski, G. S. Smith, K. Shin, S. Satija, and B. M. Ocko Langmuir 2002; 18(9) pp 3754-3757.
37 Nilsson, J., Persson, B., and von Heijne, G. (2005) Comparative analysis of amino acid distributions in integral membrane proteins from 107 genomes, Proteins 60, 606-616.
38 Wallin, E., and von Heijne, G. (1998) Genome-wide analysis of integral membrane proteins from eubacterial, archaean, and eukaryotic organisms, Protein Sci. 7, 1029-1038.
39 Jones, D. T. (1998) Do transmembrane protein superfolds exist?, FEBS Lett. 423, 281-285.
40 Seddon, A. M., Curnow, P., and Booth, P. J. (2004) Membrane proteins, lipids and detergents: not just a soap opera, Biochim. Biophys. Acta 1666, 105-117.
41 Morrissey, J. H. (2001) in Hemostasis and Thrombosis: Basic Principles and Clinical Practice (Colman, R., Hirsh, J., Marder, V., Clowes, A., and George, J., Eds.) pp 89-101, Lippincott Williams and Wilkins.
42 Morrissey, J. H., Neuenschwander, P. F., Huang, Q., McCallum, C. D., Su, B., and Johnson, A. E. (1997) Factor VIIa-tissue factor: functional importance of protein-membrane interactions, Thromb. Haemost. 78, 112-116.
43 Rezaie, A. R., Fiore, M. M., Neuenschwander, P. F., Esmon, C. T., and Morrissey, J. H. (1992) Expression and purification of a soluble tissue factor fusion protein with an epitope for an unusual calcium-dependent antibody, Protein Expr. Purif. 3, 453-460.
44 Linkins, L. A., and Weitz, J. I. (2005) New anticoagulant therapy, Annu. Rev. Med. 56, 63-77.
45 Lazarus, R. A., Olivero, A. G., Eigenbrot, C., and Kirchhofer, D. (2004) Inhibitors of Tissue Factor*Factor VIIa for anticoagulant therapy, Curr. Med. Chem. 11, 2275-2290.
46 Kubalek, E. W., Le Grice, S. F., and Brown, P. O. (1994) Two-dimensional crystallization of histidine-tagged, HIV-1 reverse transcriptase promoted by a novel nickel-chelating lipid, J. Struct. Biol. 113, 117-123.
47 Chikh, G. G., Li, W. M., Schutze-Redelmeier, M. P., Meunier, J. C., and Bally, M. B. (2002) Attaching histidine-tagged peptides and proteins to lipid-based carriers through use of metal-ion-chelating lipids, Biochim. Biophys. Acta 1567, 204-212.
48 Groves, J. T., and Dustin, M. L. (2003) Supported planar bilayers in studies on immune cell adhesion and communication, J Immunol. Methods 278, 19-32.
49 Morrissey, J. H., Fair, D. S., and Edgington, T. S. (1988) Monoclonal antibody analysis of purified and cell-associated tissue factor, Thromb. Res. 52, 247-261.
50 Nakagaki, T., Foster, D. C., Berkner, K. L., and Kisiel, W. (1991) Initiation of the extrinsic pathway of blood coagulation: evidence for the tissue factor dependent autoactivation of human coagulation factor VII, Biochemistry 30, 10819-10824.
51 Gemmell, C. H., Broze, G. J., Jr., Turitto, V. T., and Nemerson, Y. (1990) Utilization of a continuous flow reactor to study the lipoprotein-associated coagulation inhibitor (LACI) that inhibits tissue factor, Blood 76, 2266-2271.
52 Repke, D., Gemmell, C. H., Guha, A., Turitto, V. T., Broze, G. J., Jr., and Nemerson, Y. (1990) Hemophilia as a defect of the tissue factor pathway of blood coagulation: effect of factors VIII and IX on factor X activation in a continuous-flow reactor, Proc. Natl. Acad. Sci. U.S.A 87, 7623-7627.

1. A thromboplastin reagent, comprising:
(i) activated sTF,
(ii) a metal-chelating lipid,
(iii) a metal ion selected from the group consisting of Ni²⁺, Cu²⁺, Co²⁺ and mixtures thereof, and
(iv) a phospholipid.

2. The thromboplastin reagent of claim 1, wherein the activated sTF comprises an extracellular domain of TF and an oligohistidine moiety having at least 2 histidine residues.

3. The thromboplastin reagent of claim 2, wherein the activated sTF is selected from the group consisting of sTF(His)₆, sTF-5AA-(His)₆, and sTF-2(His)₅.

4. The thromboplastin reagent of claim 2, further comprising (v) Ca²⁺.

5. The thromboplastin reagent of claim 2, further comprising (vi) factor VII.

6. The thromboplastin reagent of claim 2, further comprising (vii) at least one alkali metal salt.

7. The thromboplastin reagent of claim 2, wherein the metal-chelating lipid is NTA-DOGS.

8. The thromboplastin reagent of claim 2, wherein the phospholipids comprises PS.

9. The thromboplastin reagent of claim 2, wherein the oligohistidine moiety comprises (His)ₙ, wherein n is 2-10.

10. The thromboplastin reagent of claim 2, further comprising:
(iv) Ca²⁺,
(v) factor VII, and
(vi) at least one alkali metal salt,
wherein the metal-chelating lipid is NTA-DOGS, and
the phospholipids comprises PC and PS.

11. An aPTT reagent, comprising:
(i) a metal-chelating agent,
(ii) a metal ion selected from the group consisting of Ni²⁺, Cu²⁺, and mixtures thereof, and
(iii) a phospholipid.

12. The aPTT reagent of claim 11, wherein the metal-chelating agent is a metal-chelating lipid.

13. The aPTT reagent of claim 11, further comprising Ca²⁺.

14. The aPTT reagent of claim 12, wherein the metal-chelating lipid is NTA-DOGS.

15. Activated sTF.

16. The activated sTF of claim 15, comprising an extracellular domain of TF and an oligohistidine moiety having at least 2 histidine residues.

17. The activated sTF of claim 15, selected from the group consisting of sTF(His)₆, sTF-5AA-(His)₆, and sTF-2(His)₅.

18. (canceled)

19. A combination PT and aPTT test kit, comprising:
(i) activated sTF,
(ii) a metal-chelating lipid,
(iii) a metal ion selected from the group consisting of Ni²⁺, Cu²⁺, and mixtures thereof, and
(iv) a phospholipid.

20-27. (canceled)

28. A composition for promoting clotting, comprising:
(i) a metal-chelating agent,
(ii) a metal ion selected from the group consisting of Ni²⁺, Cu²⁺, Co²⁺, Zn²⁺ and mixtures thereof, and
(iii) optionally, activated sTF comprising an extracellular domain of TF and an oligohistidine moiety having at least 2 histidine residues.

29-45. (canceled)
